(19) United States
(12) Patent Application Publication
(10) Pub. No.: US 2025/0241599 A1
(43) Pub. Date: Jul. 31, 2025

(54) CLOUD-INTEGRATED SMART NANOMEMBRANE WEARABLES FOR REMOTE WIRELESS CONTINUOUS HEALTH MONITORING

(71) Applicant: Georgia Tech Research Corporation, Atlanta, GA (US)

(72) Inventors: Woon-Hong Yeo, Atlanta, GA (US); Jared Matthews, Atlanta, GA (US)

(21) Appl. No.: 19/014,531

(22) Filed: Jan. 9, 2025

Related U.S. Application Data
(60) Provisional application No. 63/625,467, filed on Jan. 26, 2024.

(51) Int. Cl. A61B 5/00; A61B 5/0205; A61B 5/024; A61B 5/257; A61B 5/268; A61B 5/282; A61B 5/332; A61B 5/352

(52) U.S. Cl. CPC A61B 5/7267; A61B 5/0022; A61B 5/0205; A61B 5/6832; A61B 5/6833; A61B 5/7278; A61B 5/02433; A61B 5/257; A61B 5/268; A61B 5/282; A61B 5/332; A61B 5/352; A61B 2562/0209; A61B 2562/164

(57) ABSTRACT

An exemplary system and method are disclosed that measures electrocardiographic (ECG) and photoplethysmographic (PPG) signals from the sternum of postpartum women using a stretchable electrode array assembly and a flexible PPG circuit assembly, respectively, and predict an estimated blood pressure using the ECG and PPG signals in real-time using machine learning models. The exemplary system and method may also determine a heart rate parameter, a respiration rate parameter, a heart rate variability parameter, and a blood oxygen saturation parameter from the measured ECG and/or PPG signals. The determined heart rate parameter, respiration rate parameter, heart rate variability parameter, blood oxygen saturation parameter, and estimated blood pressure may be outputted to a mobile device or a healthcare portal to provide a prolonged vital sign monitor for the user.

### RELATED APPLICATION

This U.S. application claims priority to, and the benefit of, U.S. Provisional Patent Application No. 63/625,467, filed Jan. 26, 2024, entitled “CLOUD-INTEGRATED SMART NANOMEMBRANE WEARABLES FOR REMOTE WIRELESS CONTINUOUS HEALTH MONITORING OF POSTPARTUM WOMEN,” which is incorporated by reference herein in its entirety.

### STATEMENT REGARDING FEDERALLY SPONSORED RESEARCH OR DEVELOPMENT

This invention was made with government support under 3R01HD089935-04S1, awarded by the National Institutes of Health, and under ECCS-2025462, awarded by the National Science Foundation. The government has certain rights in the invention.

### BACKGROUND

Noncommunicable diseases (NCD), such as obesity, diabetes, and cardiovascular disease, are defining healthcare challenges of the 21^{st} century. Medical infrastructure has been insufficient in meeting many NCD disease patient groups' intensive, long-term monitoring needs.

Wearable technologies are prevalent but are based on rigid electronics, which may be limited in clinical use to detect NCD due to unreliable data and lack of continuous measurement ability.

There would be, therefore, a benefit to improving the health monitoring systems for long-term continuous monitoring of non-communicable diseases and general health.

### SUMMARY

An exemplary system and method are disclosed for (i) an attachable sensor device having integrated electrocardiographic (ECG) and photoplethysmographic (PPG) assemblies that can provide prolonged ECG and PPG measurements at the sternum and (ii) a controller that can estimate blood pressure or other vital sign parameters of a person.

In some embodiments, the controller is configured to estimate blood pressure using the ECG and PPG signals in real-time in one or more machine-learning models. The exemplary system and method may determine a heart rate parameter, a respiration rate parameter, a heart rate variability parameter, and a blood oxygen saturation parameter from the measured ECG and/or PPG signals as the vital sign parameters. The determined heart rate parameter, respiration rate parameter, heart rate variability parameter, blood oxygen saturation parameter, and estimated blood pressure may be outputted to a mobile device or a healthcare portal to provide a prolonged vital sign monitor for the user.

The exemplary system is portable and self-containable as both its stretchable electrode array assembly (ECG) and flexible PPG circuit assembly are configured to measure ECG and PPG signals at a nonperipheral portion of the body that relies on a PPG clipping mechanism (e.g., finger clipping) or cable connection to external devices. The stretchable electrode array assembly is configured, in some embodiments, with serpentine-structured electrodes to stretch and conform to the skin for accurate and continuous ECG measurements. The flexible PPG circuit assembly is configured with photodiodes, a PPG circuit, and a forcing membrane to accurately and continuously measure PPG signals with close contact with the skin without using a complex mechanism (e.g., finger clipping) as other PPG measurement systems.

In addition to portability, the exemplary system can be configured to be power-efficient to provide continuous measurements of ECG and PPG signals over an extended period of time (e.g., 24 hours or more) without battery replacement. The exemplary system can be used for continuous and remote ambulatory care vital sign monitoring, vital sign monitoring of postpartum women's health, or vital sign monitoring during general exercise and training, among other uses.

In an aspect, a system (e.g., without arterial line and pressure cuff) is disclosed comprising an elastomeric substrate having a first side configured to be placed in contact with a skin region of a user (e.g., having a size to place to a substantial portion of a sternum) for a period of time of at least one day, the elastomeric substrate formed of one or more layers and having defined a sensor port at a first sensor region; a flexible photoplethysmographic circuit assembly configured with photodiodes, a photoplethysmographic circuit, and a forcing membrane, the flexible photoplethysmographic circuit assembly being configured to measure photoplethysmographic (PPG), wherein the flexible circuit assembly is positioned on the elastomeric substrate such that the photodiodes are contact-able to the skin region at the sensor port for the sensor region, and wherein the forcing membrane is in mechanical contact (e.g., directly or indirectly over a tuning spring) and positioned over the photodiodes to urge the photodiodes toward the skin region; a stretchable electrode array assembly formed in the elastomeric substrate including a stretchable electrode array, configured to contact the skin region of the user at a second sensor region, the stretchable electrode array assembly being configured to measure electrocardiographic (ECG), wherein the electrode array is formed by one or more conformable electrodes having a meandering pattern configured to be in a first meandering configuration when placed on the skin and a second meandering configuration when stretched from the first meandering configuration; and a controller operatively coupled to the flexible photoplethysmographic circuit assembly and the stretchable electrode array assembly (e.g., directly or through a network), the controller having a processor; and a memory having instructions stored thereon, wherein execution of the instructions causes the processor to: receive, by the processor, measured ECG and PPG signals; determine, via a trained AI model, estimated blood pressure for the period of time of at least one day using the measured ECG and PPG signals as input to the trained AI model; and determine heart rate parameter, respiration rate parameter, heart rate variability parameter, and blood oxygen saturation parameter, from the measured ECG and/or PPG signals for the period of time of at least one day, wherein the determined heart rate parameter, respiration rate parameter, heart rate variability parameter, blood oxygen saturation parameter, and estimated blood pressure are outputted to provide a prolonged vital sign monitor for the user for the period of time of at least one day.

In some embodiments, the controller is physically coupled to the electrode array assembly and the flexible photoplethysmographic circuit in a single integrated sensor-controller device.

In some embodiments, the controller is implemented in a mobile device (e.g., tablet, smartphone) including a network interface configured to communicatively operate with the electrode array assembly and the flexible photoplethysmographic circuit through a network.

In some embodiments, the controller is a remote computing device located in a cloud infrastructure including a network interface configured to communicatively operate with the electrode array assembly and the flexible photoplethysmographic circuit through a network.

In some embodiments, the first side of the elastomeric substrate has an adhesive.

In some embodiments, the one or more conformable electrodes are formed of (i) a serpentine-patterned structure at a first end and (ii) a terminal at a second end.

In some embodiments, each serpentine-patterned structure of the one or more conformable electrodes is formed of a first layer including a metal and a second layer including a polyimide.

In some embodiments, the determined heart rate parameter, respiration rate parameter, heart rate variability parameter, blood oxygen saturation parameter, and estimated blood pressure are employed for ambulatory care monitoring.

In some embodiments, the determined heart rate parameter, respiration rate parameter, heart rate variability parameter, blood oxygen saturation parameter, and estimated blood pressure are employed for health monitoring (e.g., postpartum monitoring).

In some embodiments, the trained AI model includes a neural network.

In some embodiments, the respiration rate parameter is determined from an amplitude modulation operation of R-peaks in the measured ECG signal.

In another aspect, a method is disclosed comprising receiving, by a processor, measured ECG and PPG signals from a stretchable electrode array assembly and a flexible photoplethysmographic circuit assembly, respectively; determining, via a trained AI model, estimated blood pressure for a period of time of at least one day using the measured ECG and PPG signals as input to the trained AI model; and determining heart rate parameter, respiration rate parameter, heart rate variability parameter, and blood oxygen saturation parameter, from the measured ECG and/or PPG signals for the period of time of at least one day, wherein the determined heart rate parameter, respiration rate parameter, heart rate variability parameter, blood oxygen saturation parameter, and estimated blood pressure are outputted to provide a prolonged vital sign monitor for a user for the period of time of at least one day.

In some embodiments, one or more conformable electrodes of the stretchable electrode array assembly are formed of (i) a serpentine-patterned structure at a first end and (ii) a terminal at a second end.

In some embodiments, each serpentine-patterned structure of the one or more conformable electrodes is formed of a first layer including a metal and a second layer including a polyimide.

In some embodiments, the determined heart rate parameter, respiration rate parameter, heart rate variability parameter, blood oxygen saturation parameter, and estimated blood pressure are employed for ambulatory care monitoring.

In some embodiments, the determined heart rate parameter, respiration rate parameter, heart rate variability parameter, blood oxygen saturation parameter, and estimated blood pressure are employed for health monitoring (e.g., postpartum monitoring).

In some embodiments, the trained AI model includes a neural network.

In some embodiments, the respiration rate parameter is determined from an amplitude modulation operation of R-peaks in the measured ECG signal.

In another aspect, a non-transitory computer-readable medium is disclosed having instructions stored thereon, wherein execution of the instructions by a processor causes the processor to receive, by a processor, measured ECG and PPG signals, measured ECG and PPG signals from a stretchable electrode array assembly and a flexible photoplethysmographic circuit assembly, respectively; determine, via a trained AI model, estimated blood pressure for a period of time of at least one day using the measured ECG and PPG signals as input to the trained AI model; and determine heart rate parameter, respiration rate parameter, heart rate variability parameter, and blood oxygen saturation parameter, from the measured ECG and/or PPG signals for the period of time of at least one day, wherein the determined heart rate parameter, respiration rate parameter, heart rate variability parameter, blood oxygen saturation parameter, and estimated blood pressure are outputted to provide a prolonged vital sign monitor for a user for the period of time of at least one day.

In some embodiments, one or more conformable electrodes of the stretchable electrode array assembly are formed of (i) a serpentine-patterned structure at a first end and (ii) a terminal at a second end.

### BRIEF DESCRIPTION OF DRAWINGS

FIGS. 1A-1C each show an example system configured with (i) an attachable sensor device having integrated electrocardiographic (ECG) and photoplethysmographic (PPG) assemblies that can provide prolonged ECG and PPG measurements at the sternum and (ii) a controller that can estimate blood pressure other vital sign parameters of a person, in accordance with an illustrative embodiment. The attachable sensor device has an elastomeric substrate, a flexible photoplethysmographic (PPG) circuit positioned on the elastomeric substrate, a stretchable electrode array assembly formed in the elastomeric substrate, and a controller coupled to the flexible PPG circuit assembly and the stretchable electrode array assembly. FIG. 1A operates the controller on the attachable sensor device, wherein the controller is physically coupled to the electrode array assembly and the flexible PPG circuit. FIG. 1B implements the controller on an analysis/diagnosis application in a mobile device (e.g., tablet, smartphone) comprising a network interface configured to communicate with the electrode array assembly and the flexible PPG circuit through a network. FIG. 1C operates the controller on a remote computing device located in a cloud infrastructure comprising a network interface configured to communicate with the electrode array assembly and the flexible PPG circuit through a network.

FIG. 2 shows an example operation flow for the exemplary system of FIG. 1A-1C in accordance with an illustrative embodiment.

FIGS. 3A-3H show further details of the example attachable sensor device of the exemplary system of FIGS. 1A-1C and an example fabrication process for the same. FIGS. 3A-3C show the exemplary sensor device configured with a soft bilayer patch, nanomembrane electrode, and flexible electronics to monitor postpartum women's health. FIG. 3D shows an example flexible photoplethysmographic (PPG) circuit assembly configured with (i) a photoplethysmographic circuit having an infrared LED and photodiodes and (ii) a forcing membrane. FIG. 3D shows the fabrication process for the sensor device. FIGS. 3F-3H show an example cloud-integrated application employed by the exemplary system and the end-to-end functionality of the exemplary system.

FIGS. 4A-4Q show a fabricated sensor device of the exemplary system and the associated analysis and experiment for the fabricated sensor device. FIGS. 4A-4F show the fabricated sensor device configured with a soft bilayer patch, nanomembrane electrode, and flexible electronics. FIGS. 4G-4L show the finite element analysis of the electrode pattern in the fabricated sensor device. FIGS. 4M-4O show the sensing characterization of the fabricated sensor device. FIGS. 4P-4Q show the oxygen desaturation time experiment for the fabricated sensor device.

FIGS. 5A-5X show the experiment protocols and at-home performance and mechanical evaluation for the exemplary system. FIGS. 5A-5I show the performance and evaluation of the exemplary system on 20 participants over 28 days. FIGS. 5J-5O show remote clinical results from the exemplary system for differentiating risk levels. FIGS. 5P-5U show a machine learning (ML) model configured for the exemplary system. FIG. 5V shows a machine learning preprocessing, training, and evaluation pipeline for the exemplary system. FIG. 5W shows the synchronized waveform processing for machine learning, which shows ABP, ECG, and PPG. FIG. 5X shows the distribution of PPG and ECG scalogram values generated after transforming the input dataset for the machine learning pipeline (i.e., ML model).

### DETAILED DESCRIPTION

Some references, which may include various patents, patent applications, and publications, are cited in a reference list and discussed in the disclosure provided herein. The citation and/or discussion of such references is provided merely to clarify the description of the disclosed technology and is not an admission that any such reference is “prior art” to any aspects of the disclosed technology described herein. In terms of notation, “[n]” corresponds to the nth reference in the list. For example, [1] refers to the first reference in the list. All references cited and discussed in this specification are incorporated herein by reference in their entirety and to the same extent as if each reference was individually incorporated by reference.

### Example System

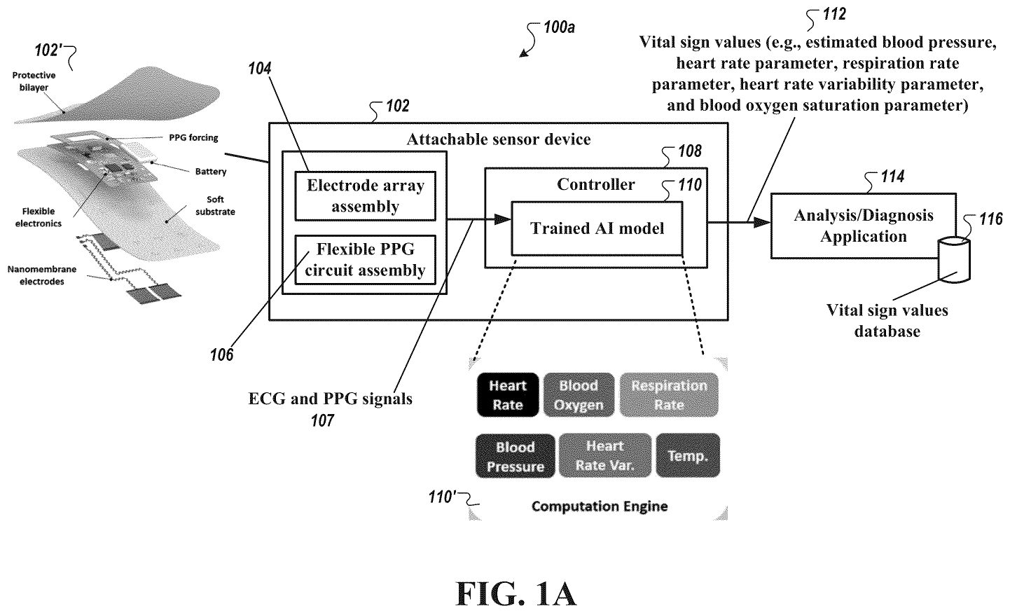

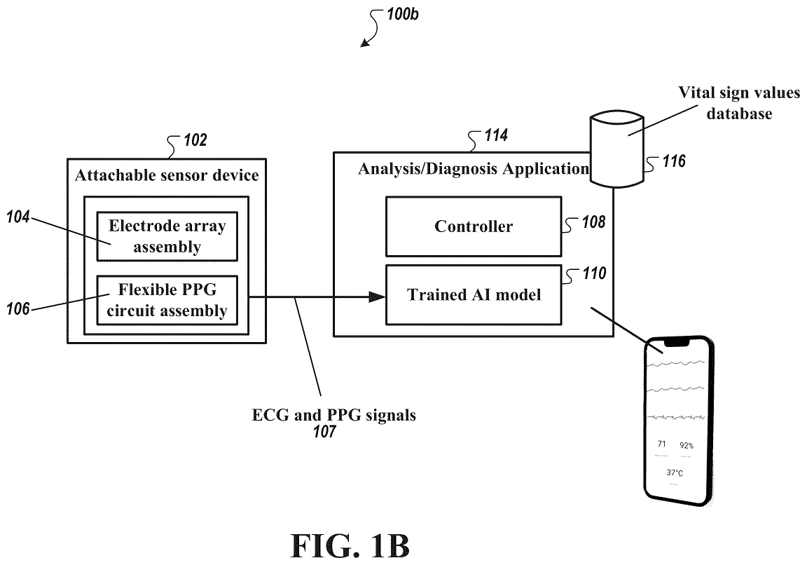

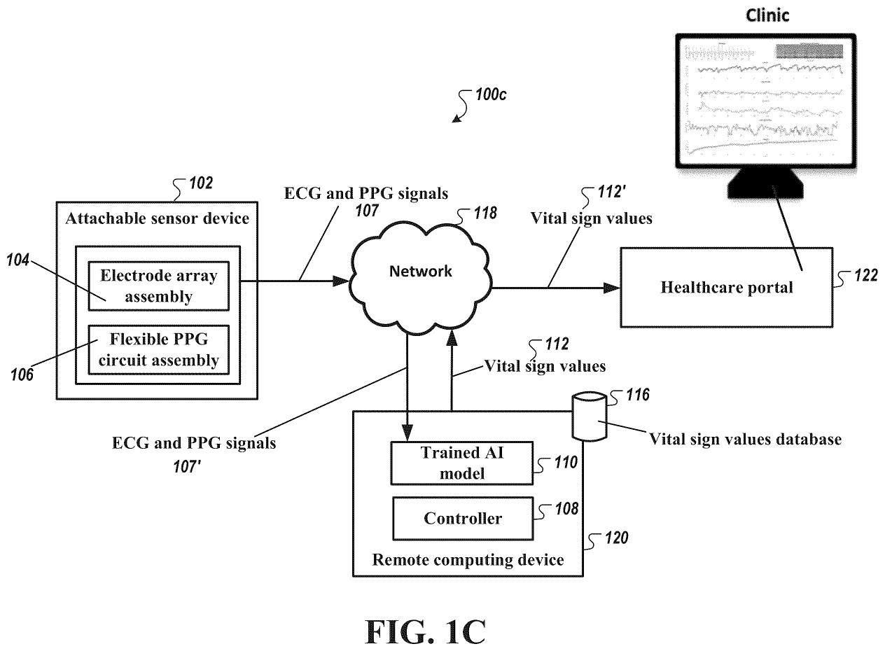

FIGS. 1A-1C each shows an example system 100 (shown as 100a, 100b, 100c) configured with (i) an attachable sensor device having integrated electrocardiographic (ECG) and photoplethysmographic (PPG) assemblies that can provide prolonged ECG and PPG measurements at the sternum and (ii) a controller that can estimate blood pressure other vital sign parameters of a person, in accordance with an illustrative embodiment. FIG. 1A operates the controller on the attachable sensor device, wherein the controller is physically coupled to the electrode array assembly and the flexible PPG circuit. FIG. 1B implements the controller on an analysis/diagnosis application in a mobile device (e.g., tablet, smartphone) comprising a network interface configured to communicate with the electrode array assembly and the flexible PPG circuit through a network. FIG. 1C operates the controller on a remote computing device located in a cloud infrastructure comprising a network interface configured to communicate with the electrode array assembly and the flexible PPG circuit through a network.

In the examples shown in FIGS. 1A-1C, the systems 100a-100c each includes an attachable sensor device 102 (shown as 102′), wherein the attachable sensor device 102 is configured with the elastomeric substrate (not shown), the stretchable electrode array assembly 104 and the flexible PPG circuit assembly 106. The elastomeric substrate (not shown) may have a first side configured to be placed in contact with a skin region of a user (e.g., having a size to place a substantial portion of a sternum) for a period of time of at least one day. The elastomeric substrate is formed of one or more layers having defined a sensor port at a first sensor region. Additionally, the first side of the elastomeric substrate may have an adhesive.

The electrode array assembly 104 may be formed in the elastomeric substrate comprising a stretchable electrode array, configured to contact the skin region of the user at a second sensor region. The electrode array assembly 104 is configured to measure electrocardiographic (ECG), wherein the electrode array is formed by one or more conformable electrodes having a meandering pattern configured to be in a first meandering configuration when placed on the skin and a second meandering configuration when stretched from the first meandering configuration. The one or more conformable electrodes (not shown) of the electrode array assembly 104 may be formed of (i) a serpentine-patterned structure at a first end and (ii) a terminal at a second end. Each serpentine-patterned structure may be formed of a first layer comprising a metal and a second layer comprising a polyimide.

The flexible PPG circuit assembly 106 is configured with photodiodes, a PPG circuit, and a forcing membrane. The flexible PPG circuit assembly 106 is configured to measure photoplethysmographic (PPG), wherein the flexible circuit assembly 106 may be positioned on the elastomeric substrate such that the photodiodes are contact-able to the skin region at the sensor port for the sensor region, and wherein the forcing membrane is in mechanical contact (e.g., directly or indirectly over a tuning spring) and positioned over the photodiodes to urge the photodiodes toward the skin region.

The systems 100a-100c each also includes a controller 108 operatively coupled to the electrode array assembly 104 and the flexible PPG circuit assembly 106, either directly (FIGS. 1A-1B) or through a network (FIG. 1C). The controller 108 is configured to (i) receive, via a processor, measured ECG and PPG signals 107 (shown as 107′) from the electrode array assembly 104 and flexible PPG circuit assembly 106, (ii) determine, via a trained AI model 110 (shown as 110′), estimated blood pressure for the period of time of at least one day using the measured ECG and PPG signals 107 as input to the trained AI model 110, and (iii) determine heart rate, respiration rate, heart rate variability, and blood oxygen saturation parameters from the measured ECG and/or PPG signals for the period of time of at least one day. The estimated blood pressure and the determined heart rate, respiration rate, heart rate variability, and blood oxygen saturation parameters are shown as vital sign values 112. Additionally, the trained AI model 110 may comprise a neural network, and the respiration rate may be determined from an amplitude modulation operation of R-peaks in the measured ECG signal.

The vital sign values 112 (shown as 112′) may be outputted on the analysis/diagnosis application 114 (FIGS. 1A-1B) or the remote computing device 120 (via the network 118) (FIG. 1C) to provide a prolonged vital sign monitor for the period of time of at least one day. The vital sign values 112 may be subsequently stored in a vital sign values database 116 associated with the application 114 (FIGS. 1A-1B) or the computing device 120 (FIG. 1C) for ambulatory care monitoring or health monitoring (e.g., postpartum monitoring).

In the example shown in FIG. 1C, the system 100c further comprises a healthcare portal 122, wherein the healthcare portal 122 is configured to receive, via the network 118, the vital sign values 112′. The vital sign values 112′, as aforementioned, may be employed (e.g., by clinics and hospitals) for real-time remote health monitoring.

### Example Method

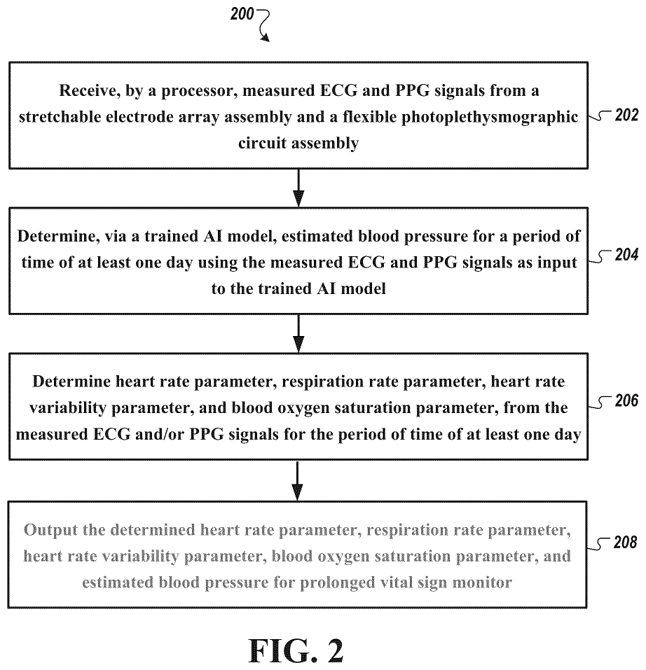

FIG. 2 shows an example operation flow 200 for the exemplary system.

At step 202, the exemplary system may receive, by a processor, measured ECG and PPG signals from a stretchable electrode array assembly and a flexible photoplethysmographic circuit assembly, respectively.

At step 204, the exemplary system may determine, via a trained AI model, estimated blood pressure for a period of time of at least one day using the measured ECG and PPG signals as input to the trained AI model.

At step 206, the exemplary system may determine heart rate parameter, respiration rate parameter, heart rate variability parameter, and blood oxygen saturation parameter, from the measured ECG and/or PPG signals for the period of time of at least one day.

At step 208, the exemplary system may output the determined heart rate parameter, respiration rate parameter, heart rate variability parameter, blood oxygen saturation parameter, and estimated blood pressure for prolonged vital sign monitor.

### Example Health Monitoring System

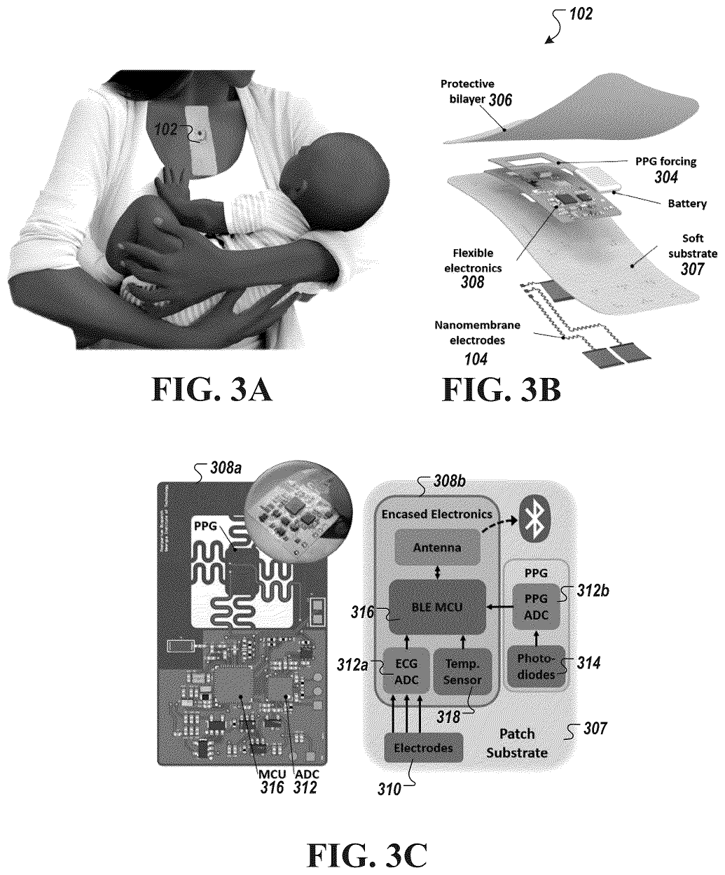

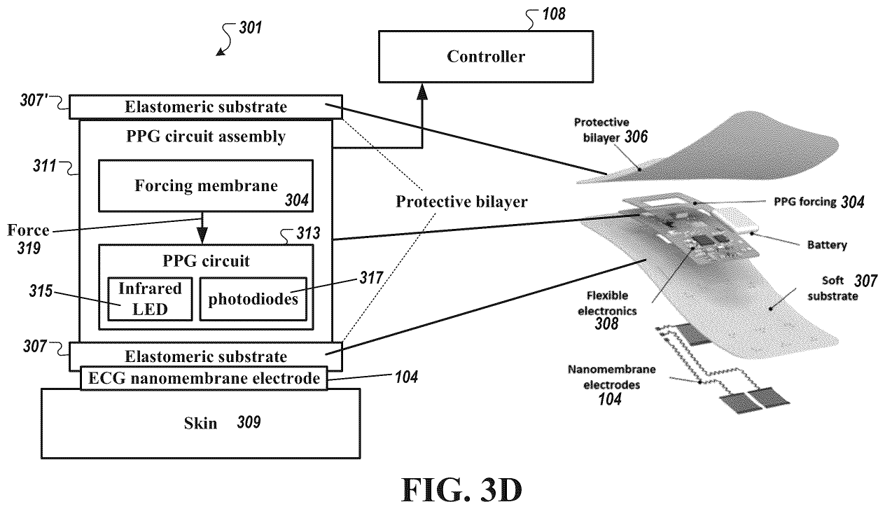

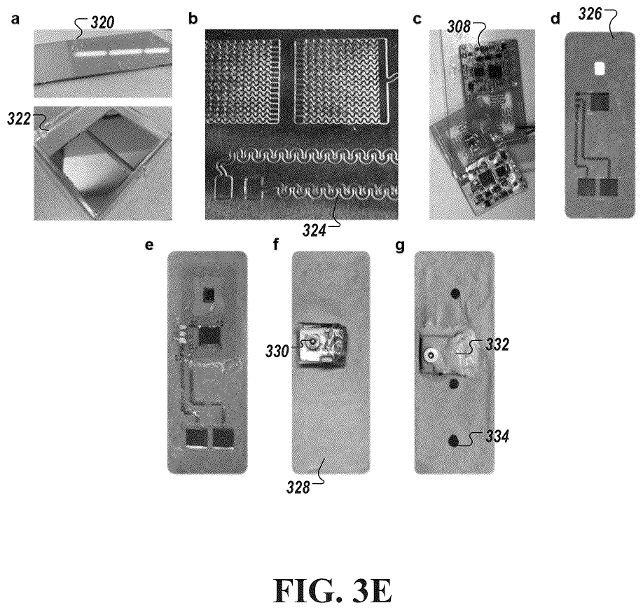

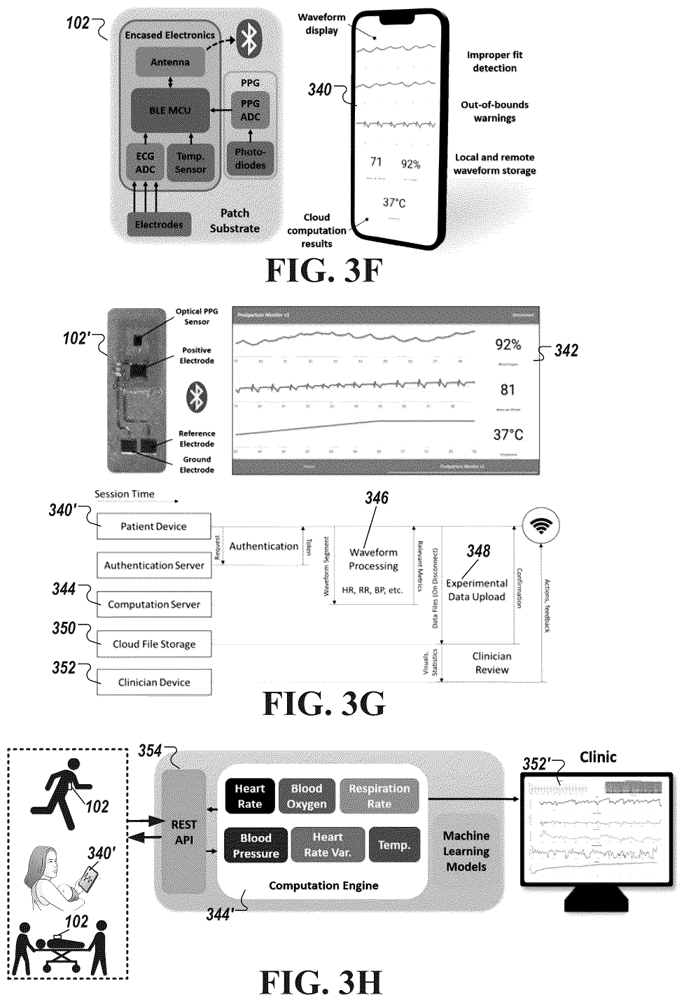

The exemplary system employs a cloud-integrated mobile application, associated cloud architecture, and a wireless patch. FIGS. 3A-3C show an example attachable sensor device of the exemplary system configured with a soft bilayer patch, nanomembrane electrode, and flexible electronics to monitor postpartum women's health.

The attachable sensor 102 (i.e., nanomembrane electrodes in a bilayer patch) may be attached to a woman's sternum, as shown in FIG. 3A. Electrocardiographic (ECG) and photoplethysmography (PPG), the key signals measured by the attachable sensor device 302, may provide insight into cardiovascular health through their waveform morphology and from the derivation of key metrics such as heart rate, blood oxygen saturation, and heart rate variability.

As shown in FIG. 3B, a flexible passive forcing mechanism, along with the attachable sensor's other key components, may be developed to improve PPG contact pressure 304 (i.e., PPG forcing) at the sternum, which may enhance the quality of signal at this otherwise challenging PPG sensing location [13e]. An encapsulating bilayer design 306 projects this mechanism and other internal components (e.g., 308) from damage without compromising the flexibility or softness of the sensor. Within this bilayer 306, flexible electronics 308 (shown as 308a, 308b in FIG. 3C, shown as 102 in FIGS. 1A-1C) can provide ECG using electrodes 310 and ECG analog-to-digital converter 312a (ADC), provide PPG using photodiodes 314 and PPG ADC 312b, and provide temperature sensing using temperature sensor 318, as shown in FIG. 3C.

FIG. 3D shows an example flexible photoplethysmographic (PPG) circuit assembly 301, shown as 106 (part of sensor device 102) in FIGS. 1A-1C. As shown, the flexible PPG circuit assembly 301 is configured with (i) a photoplethysmographic circuit 313 having an infrared LED 315 and photodiodes 317 and (ii) a forcing membrane 304 (referred to as PPG forcing 304), though other types of light sources and corresponding photodiodes may be used in various aspects.

In the PPG circuit assembly 301, the PPG circuit 313 is configured to measure PPG by emitting a low-intensity infrared ray through biological tissues (e.g., skin) using the infrared LED 315. The infrared ray may be absorbed by the bone and blood vessels; only a fraction of the infrared light may be reflected back to the photodiodes 317 that may be used to measure the PPG signals.

The PPG circuit assembly 301 is placed on an elastomeric substrate 307 (referred to as soft substrate 307), wherein the elastomeric substrate 307 has a first side configured to be placed in contact with a skin region 309 of a user for a period of time of at least one day. The elastomeric substrate 307 may be formed of one or more layers and has defined a sensor port at a first sensor region. The PPG circuit assembly 301 is positioned on the elastomeric substrate 307 in a way that the photodiodes 317 are contact-able to the skin region at the sensor port for the sensor region, and wherein the forcing membrane 304 is in mechanical contact (e.g., directly or indirectly over a tuning spring) and positioned over the photodiodes 317 to urge the photodiodes toward the skin region with a force 319. The PPG circuit assembly 311 is also covered by another elastomeric substrate 307′. The elastomeric substrates 307 and 307′ may form a protective bilayer 306 over the PPG circuit assembly 311.

The stretchable electrode array assembly 104 (shown as nanomembrane electrodes) is formed in the elastomeric substrate 107 and configured to contact the skin region 109 of the user to measure the ECG signals. The stretchable electrode array assembly 104 and the PPG circuit assembly 311 together form the sensor device 102 shown in FIGS. 1A-1C. Additionally, the controller 108 is operatively coupled to the stretchable electrode array assembly 104 and the flexible PPG circuit assembly 311 (e.g., directly or through a network).

FIG. 3E shows an example fabrication process for the attachable sensor device of the exemplary system. As shown in step (a), a polyimide film 320 is placed on a glass slide 322 spin-coated with polydimethylsiloxane (PDMS). Copper, chromium, and gold layers are deposited into the polyimide film via electron beam evaporation. In step (b), an electrode pattern 324 is extracted from a plated slide, produced from the electron beam evaporation shown in step (a), using a femtosecond pulse duration laser. In step (c), a circuit 308 (i.e., flexible electronics) is manufactured and programmed. In step (d), the electrode pattern 324 is transferred to a patch substrate 326, which is a medical tape with skin-safe adhesive coating cut to a desired profile. In step (e), the circuit 308 is installed via epoxy, and the electrode pattern 324 is attached to the circuit with anisotropic conducting film. In step (f), a battery 330 and the protective bilayer 328 are installed on the other side of the patch substrate. In step (g), the battery 330 and circuit 308 are encapsulated by plastic 332 (e.g., Ecoflex), with sensors marked (e.g., 334) for ease of patient use.

FIGS. 3F-3H show an example cloud-integrated application employed by the exemplary system and end-to-end functionality of the exemplary system. In one instance, as shown in FIG. 3F, the attachable sensor device 102 (shown as 102′) may implement the Bluetooth Low-Energy (BLE) communication protocol for the wireless delivery of biosignal data to a mobile device (e.g., tablet, smartphone) with a custom mobile application 340 (shown as 340′) targeting multiple operating systems to ensure uniform compatibility with 90% of US consumers with a mobile device of any kind [15], a coverage that remains uncommon in other wearable systems.

As shown in FIG. 3G, the mobile application 340 may serve multiple functions in the exemplary system: the application may (i) interface with the soft device 102′ to receive the raw waveform data 342, (ii) provide segments of these waveforms to a cloud-based computation engine 344 (shown as computation server 344′) to process (at 346), (iii) save the waveform data (at 348) to the cloud 350 (shown as cloud file storage) for offline processing, and (iv) further save this data to local device storage 352 (shown as clinician device 352′) for future uploading if an internet connection cannot be established.

The mobile application 340 may impose a constraining interface on prospective connections and reuse functionality between these connections, providing support for an ecosystem of sensor devices by collecting and processing data from potentially multiple points of measurement simultaneously. The mobile application 340 may also provide access to the modern cloud technologies that underpin the system's remote monitoring ability. Modern cloud solutions are scalable under load, deploying more servers to handle user requests in parallel. Despite being reliable and readily integrated with existing mobile software development kits, the cloud has received little attention in the wearable device field. The previous studies that include cloud technologies leverage them in an internet-of-things capacity, with direct device connections to the cloud, via the Message Queuing Telemetry Transport protocol [16]. While this may simplify the design process obtained by bypassing mobile device integration and application development, real-time user feedback may become difficult or impossible.

The exemplary system may use the cloud in two ways. First, a cloud-integrated application, as shown in FIGS. 3G-3H, may give users real-time characterization of their cardiovascular health through a low-maintenance, highly scalable computation engine 344′. The computation engine 344′ may be serverless—that is, details of its resource provisioning, maintenance, and scaling under load are handled by the cloud provider, allowing greater research effort toward device development. The engine 344′ may provide the computational power to process thousands of simultaneous waveform submissions to determine heart rate, respiration rate, blood pressure, heart rate variability, and blood oxygen saturation in real-time. These are then returned to the client's mobile device 340′ via an application programming interface 354 (e.g., Rest API) for presentation.

Cloud-based signal quality may be determined by computing signal quality indices (SQIs). The application 340′ may prompt users to correct the sensor device placement given out-of-bounds values of these SQIs as described in [17], maintaining signal quality throughout each measurement session. Beyond real-time calculations, the cloud may provide a database 350 for uploading waveform files, enabling long-term clinical monitoring and decision-making through offline clinician access to patient data. An application programming interface 354 (API) may be exposed to the patient's mobile device 340′ over the internet via an industry-standard representational state transfer interface to parse, authenticate, and distribute requests to the engine instances such that no single instance is overwhelmed and latencies for the user remain low.

Additionally, the exemplary system may continuously function with the integrated cloud, as discussed, when the user is running or in ambulatory care as long as the sensor device 102 is attached to the user's sternum.

### EXPERIMENTAL RESULTS AND ADDITIONAL EXAMPLES

A study was conducted to develop and evaluate the exemplary system and method comprising (i) an attachable sensor device having integrated electrocardiographic (ECG) and photoplethysmographic (PPG) stretchable sensors that can provide prolonged ECG and PPG measurements at the sternum and (ii) a controller that can estimate blood pressure other vital sign parameters of a person.

### Fabricated System

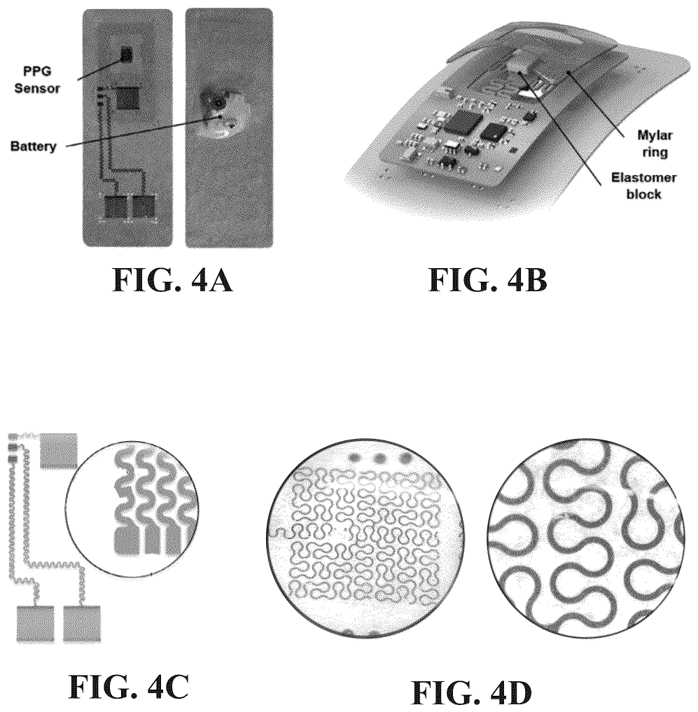

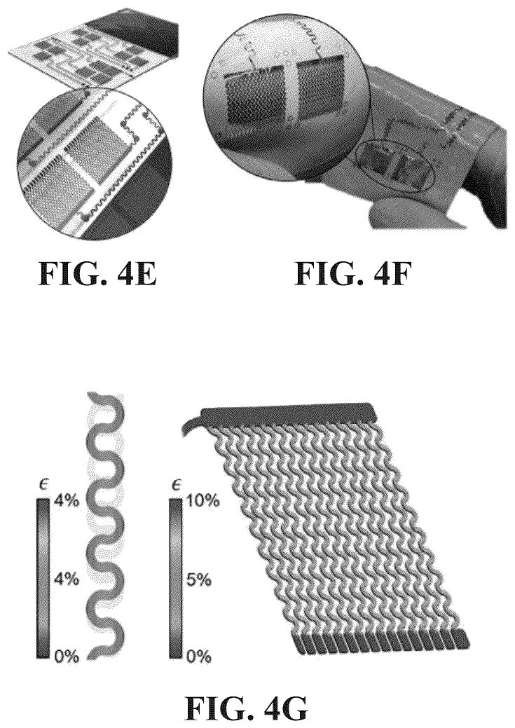

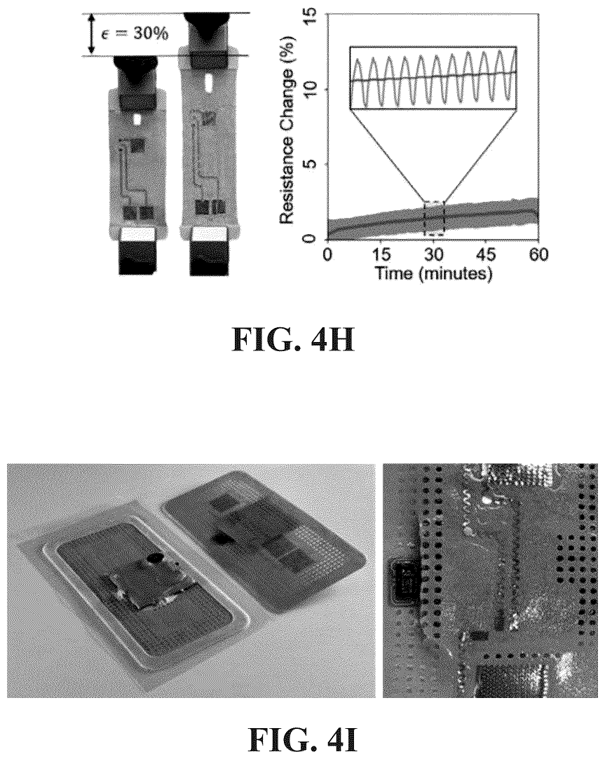

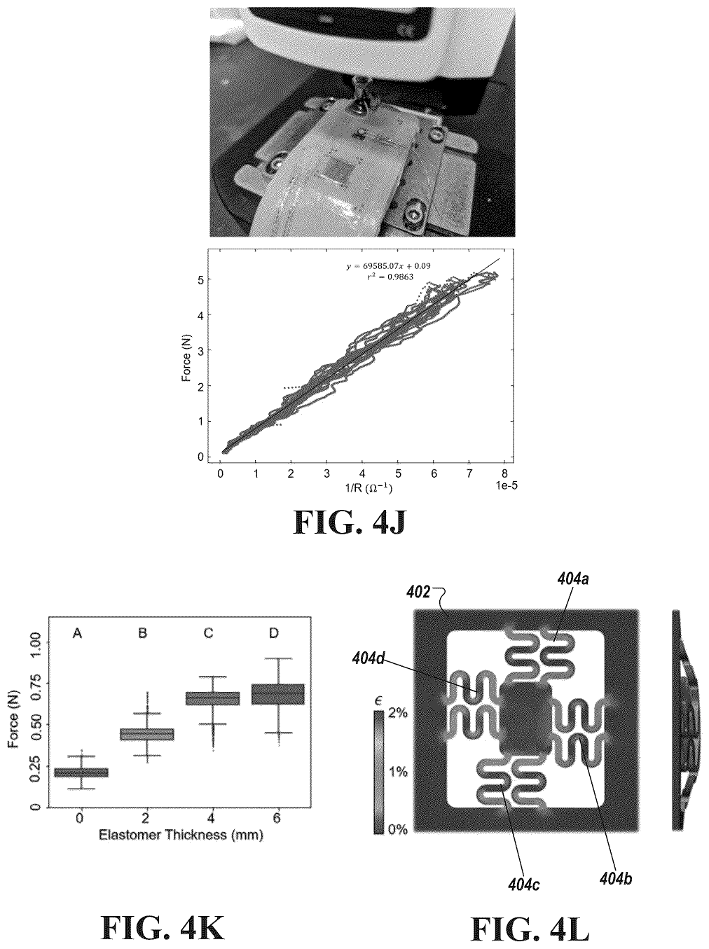

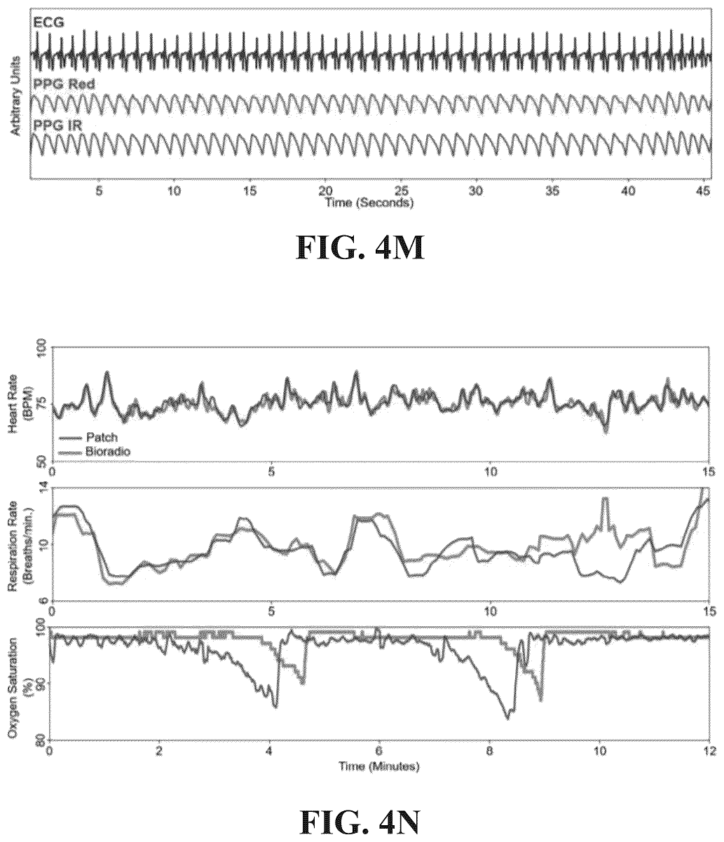

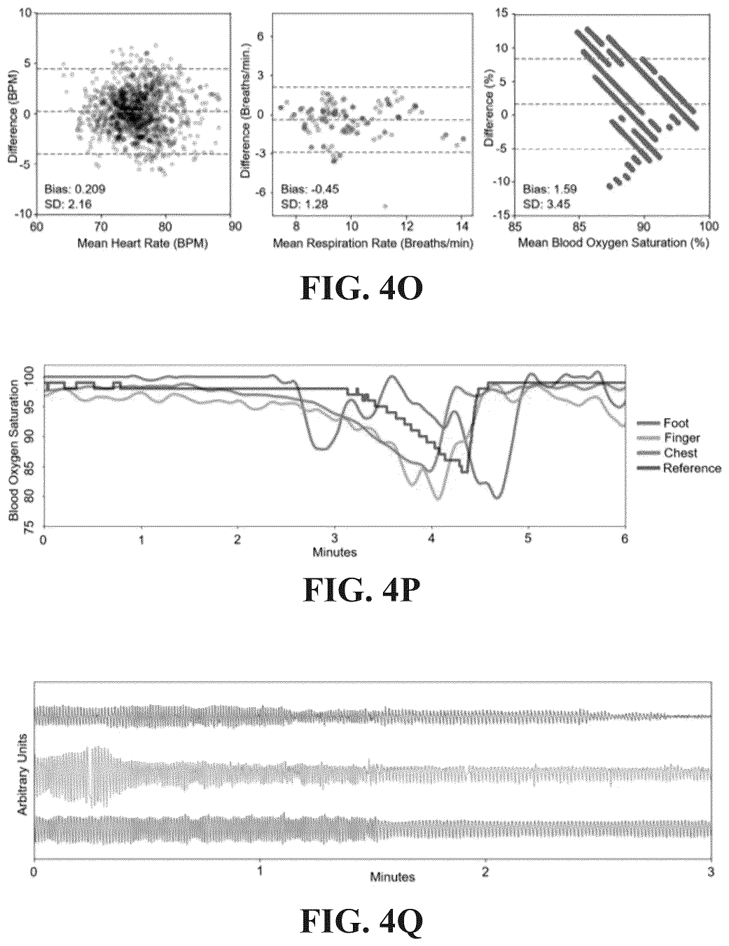

FIGS. 4A-4F show a fabricated attachable sensor device configured with a soft bilayer patch, nanomembrane electrode, and flexible electronics. Mechanical characterization of the fabricated sensor device, as shown in FIG. 4A, was necessary given its intended use in weeks-long, unaided remote monitoring applications. Specifically, the nanomembrane electrode pattern and PPG forcing mechanism, as shown in FIG. 4B, were evaluated in both simulation and laboratory testing. The electrodes were composed of copper, chromium, and gold layers of electron-beam deposited on polyimide (PI) film to provide a skin-safe, flexible form factor known to exceed the signal-to-noise ratio (SNR) obtained rigid clinical alternatives for ECG measurement. The sensing portion of the electrode, as shown in FIG. 4C, permitted long-term, unaided application and removal without electrode delamination from the patch substrate. Building on the conventional “Greek cross” fractal serpentine pattern described in [18], pairs of opposite ends of each electrode were affixed to the fabric substrate with a thin layer of skin-safe elastomer, eliminating delamination from normal forces. Avoiding this delamination was critical for long-term monitoring because, once initiated, the delamination propagated down the length of the flexible electrode and rendered the sensor device unable to sense ECG. FIG. 4D shows two such failure modes.

The study used a femtosecond pulse duration laser for extracting the pattern from the electron beam-coated PI, as the density of electrode patterns seen immediately after cutting (shown in FIG. 4E) and on the patch (shown in FIG. 4F) was not achievable with conventional pulse durations that would otherwise thermally load the substrate.

The study used finite element analysis (FEA) to explore this new electrode pattern. Uniaxial electrode strains of 15% were applied to observe the maximum local strain in the serpentine designs, both within the interconnects and the electrode pads. FIGS. 4G-4L show the finite element analysis of the electrode pattern in the fabricated sensor device.

FIG. 4G shows maximum local strains of 8% and 10% for the interconnect and sensing pattern, respectively, indicating a marginal strain reduction with this design. In addition, real-world electrode characterization, as shown in FIG. 4H, shows mechanical and electrical robustness in response to large cyclic strain (30%).

The study plotted electrode resistance for 100 cycles over the course of an hour, with average resistance increasing by 2%, within acceptable bounds for ECG collection. Previous studies indicated that measuring high-quality PPG from the sternum was feasible for long periods, given an applied force on the PPG sensor that overcame the lack of local vascularization [13e]. Indeed, early device iterations lacking deliberate forcing of the PPG unit had poor PPG collection ability at the sternum as shown in FIG. 4I. Therefore, the passive PPG forcing mechanism was developed to force the PPG against the underlying skin via an elastomer block serving as a tunable spring. A laser-cut, flexible mylar bracket was also adhered to the circuit to hold the block in place during use. Naturally, larger blocks can generate more force, but increasing the elastomer height (and therefore sensor displacement) also prevented more of the surrounding adhesive area from remaining in contact with the skin, limiting the total force ultimately imparted on the PPG in a steady state. This trade-off required optimization to find the block thickness generating the maximum force with minimal variance over replicates of the experiment.

The study found achievable PPG force for 0-, 2-, 4-, and 6-mm blocks using the experimental setup as shown in FIG. 4J. Force increased with elastomer height with approximately the same variance up to and including 4 mm, plateauing in average force with a larger variance at 6 mm. All group distributions were different from each other (p<0.001) as indicated by the letters A, B, C, and D as shown in FIG. 4K. Seeking to maximize contact pressure consistently, the 4 mm elastomer block height was selected for PPG forcing, given the higher variance of the 6 mm group and negligible difference in the median generated force of the two groups.

The study also placed the PPG sensor on a flexible printed circuit board (fPCB) 402 with serpentine connections 404a-404d such that large out-of-plane strains of the sensor from the passive forcing remained possible with no broader deformation of the fPCB or excessive local strain. FEA of the fPCB's interconnect design, as shown in FIG. 4L, confirmed that even with a high force for the PPG forcing mechanism of 1N, local interconnect strain peaked at 2.0%. All candidate elastomer block heights, as shown in FIG. 4K, reported achievable PPG force values less than what was simulated as shown in FIG. 4L, indicating the suitability of the interconnect design for the considered elastomer blocks.

### Evaluation of the Fabricated System

Multi-sensing performance. The study characterized the fabricated sensor device of the exemplary system by comparison with a clinical-grade reference device (shown as BioRadio) with conventional gel electrode ECG and finger PPG sensing. FIGS. 4M-4O show the sensing characterization of the fabricated sensor device.

FIG. 4M shows representative and aligned ECG and PPG waveforms from this controlled setting. Both the amplitude modulation of the ECG R-peaks from which the respiration rate was derived and the dichroitic notch of the PPG were visible. FIG. 4N shows the derived heart rate, respiration rate, and blood oxygen metrics. Heart rate variability, while calculated by the cloud platform for its importance in arrhythmia detection, among other use cases, was not provided by the reference device (shown as BioRadio). These measurements, central to cardiovascular health characterization and presented to both patients and clinicians, showed broad agreement with the reference measurements. This was confirmed by Bland-Altman plots for the same period as shown in FIG. 4O.

There was a lag in the blood oxygen saturation level (SpO₂) reported by the reference device (shown as BioRadio) compared to the fabricated sensor device. To determine whether this was a result of the distance from the pulmonary system to both the finger and the sternum or simply an artifact in the SpO₂ reporting from the reference device, the study carried out a follow-up experiment to compare re-saturation timing between sensors at the finger, chest, and toe.

FIGS. 4P-4Q show the oxygen desaturation time experiment. FIG. 4P shows the timing difference between chest, finger, and foot PPG sensors, with chest data providing the shortest response time. FIG. 4Q shows the corresponding waveforms at the onset of desaturation, demonstrating reduced amplitudes characteristic of hypoxic conditions at peripheral sites such as the toe.

Using the return to saturation as a shared landmark, chest PPG saw a nearly 30-second detection lead compared to finger sensors. The reference device and an identical PPG sensor (MAX30102) on the chest showed exact response times at the finger, reinforcing this finding and the previously observed lag in the reference sensor. Continuing this trend, the toe sensor was approximately 30 seconds delayed. While some disruption was likely due to subject motion during this measurement, the re-saturation point was pronounced. It made for a minute difference between the chest and toe regarding saturation detection. This had important implications for wearable systems (e.g., fabricated sensor device) designed for real-time monitoring, as this example illustrated the role of sensing location (and the sternum's optimality thereof) in a wearable device's ability to monitor the patient's physiological state with respect to oxygen saturation.

Validation of clinical feasibility. The exemplary system, comprising an attachable sensor device, mobile device and application, and cloud platform, was validated in an at-home experiment with twenty Black women during their first month postpartum. A pair of devices was provided to each participant, classified by maternal health clinicians as belonging to a high or low-risk population based on their medical history and health at discharge from the hospital. The high-risk postpartum group was defined as those individuals with any history of cardiomyopathy, diabetes, thromboembolism, hypertension, or those who had ever given birth through Cesarean section. All of these were risk factors for postpartum complications defined in the study as the occurrence of infection, thromboembolism, hypertensive crisis, cardiomyopathy, heart failure, myocardial infarction, or hospitalization, the early manifestations of which this system was designed to monitor. The high-risk experimental group contrasted with the low-risk participants, who had no history of the high-risk diseases and had no documented complications during their most recent pregnancy and subsequent childbirth, such as abnormal fetal development or Cesarean delivery.

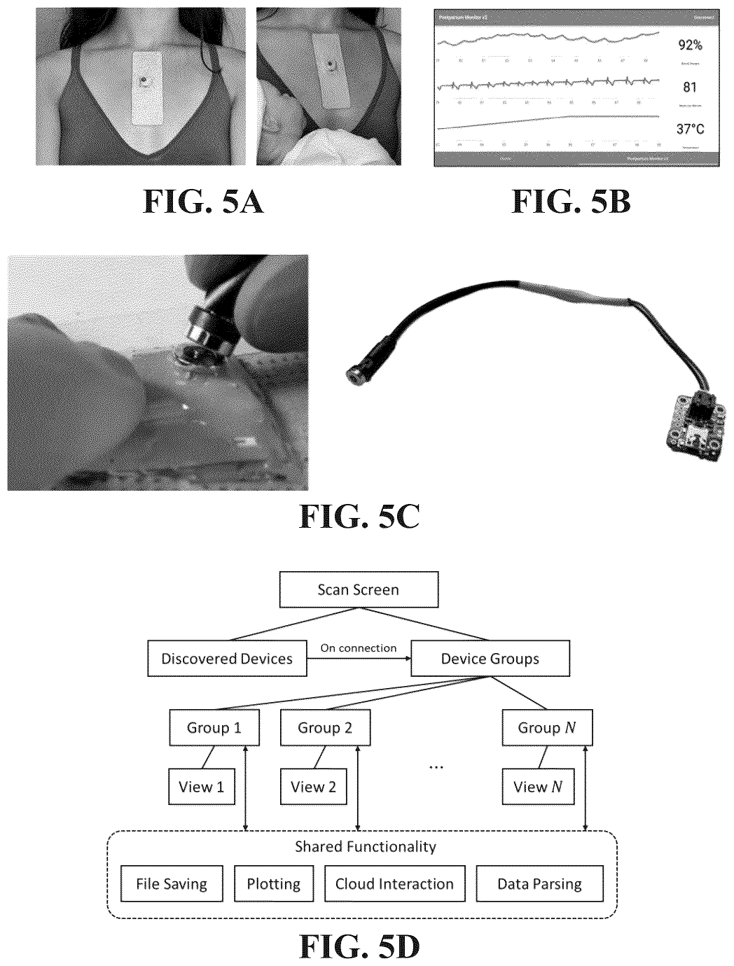

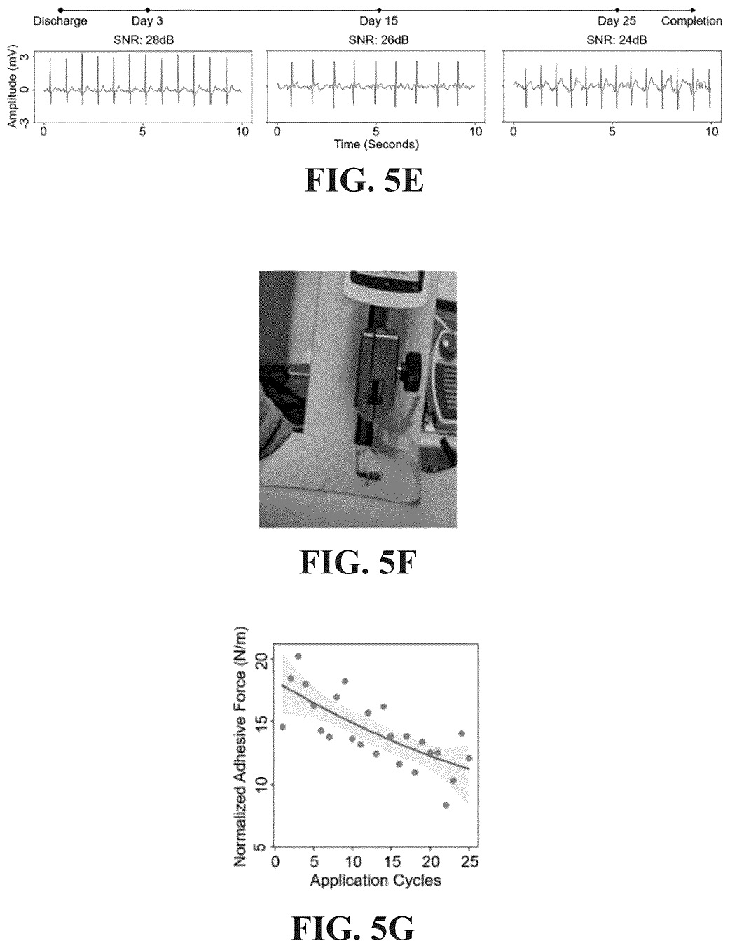

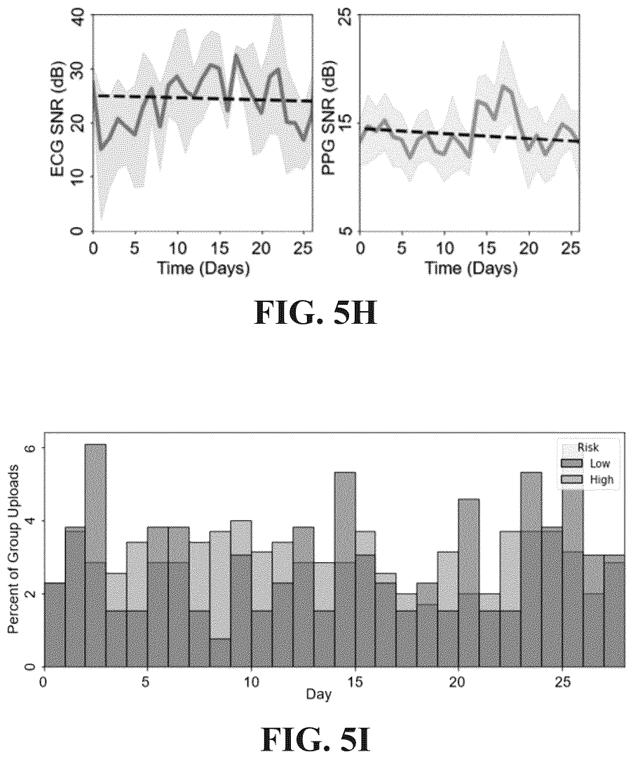

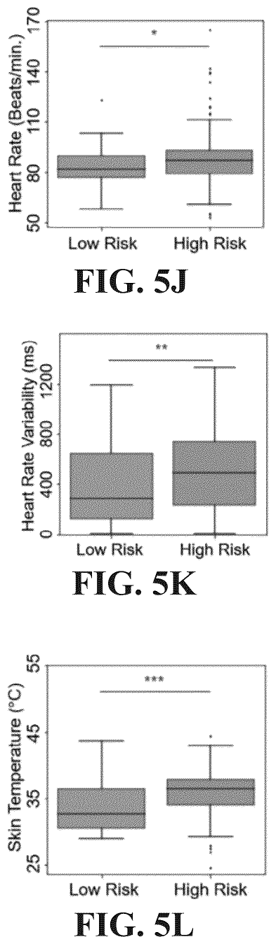

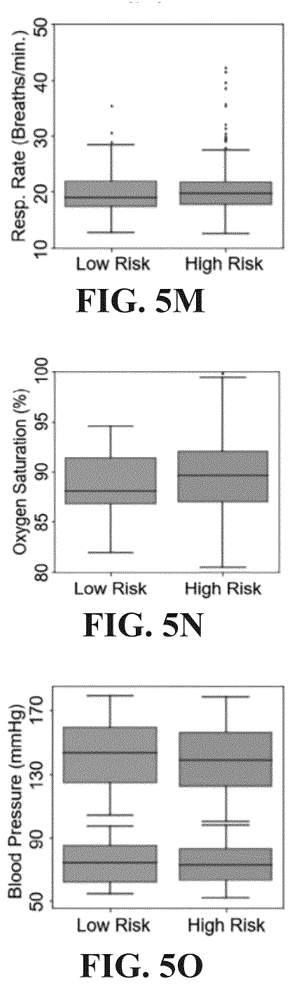

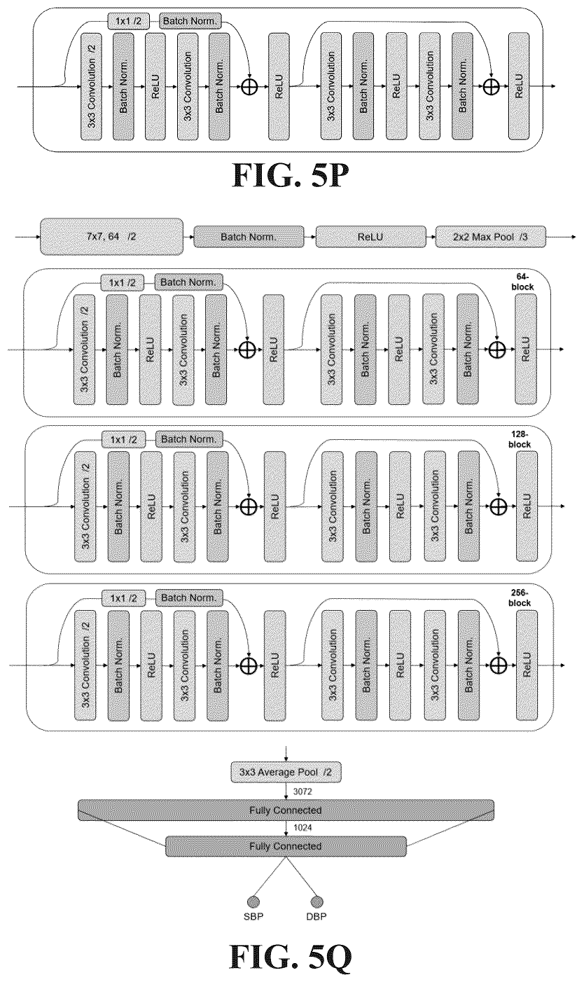

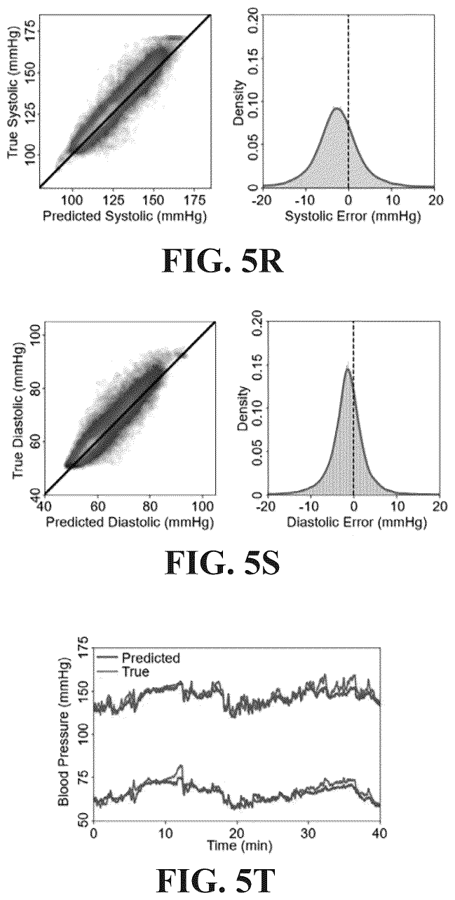

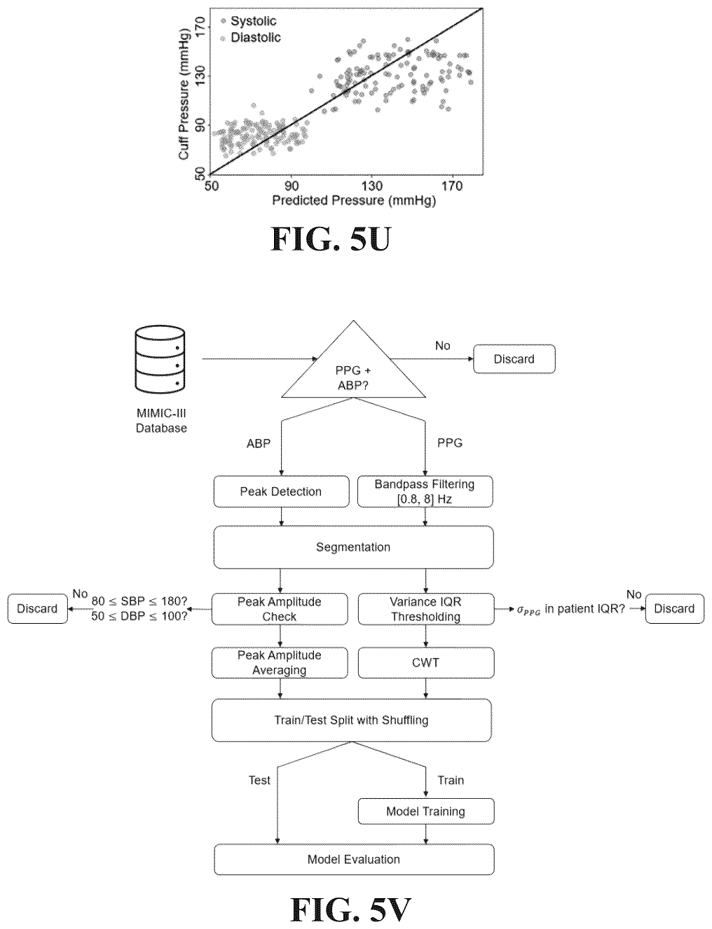

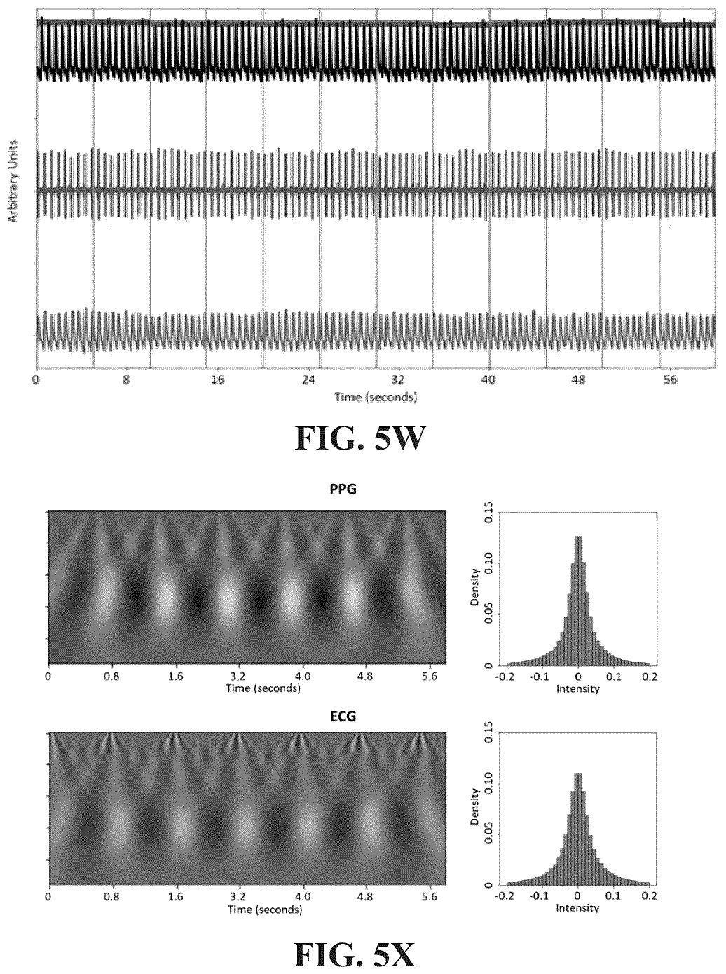

FIGS. 5A-5I show an at-home performance and mechanical evaluation of the exemplary system on 20 participants over 28 days. All participants were instructed to wear the fabricated sensor device, as shown in FIG. 5A, for a total of at least 10 minutes per day for the 28 days of the experiment, to assess the system's ability to inform both the participants and the clinicians of the former's cardiovascular health. As shown in FIG. 5B, participant ECG was used to calculate heart rate (HR), respiration rate (RR), and heart variability rate (HRV), while SpO₂ and a second HR measurement were obtained from PPG. Temperature readings were also considered by the clinic for interrogation of fever and infection.

In addition to the two sensor devices, participants were provided a USB-based magnetic charger (as shown in FIG. 5C), an Android tablet loaded with the mobile application, and an informational video explaining the usage of the device. The tablet interface provided participants with real-time feedback on heart rate, blood oxygen, and skin temperature and notified the user to adjust the fit of the device if poor data quality was detected by the cloud. FIG. 5D shows the usage of the fabricated sensor device and mobile application.

A set of data shown in FIG. 5E documented signal quality for a representative patient across the duration of the study. Though the retention of quality was not as high as the controlled testing, the quality was sufficient even on the final day of the survey for calculating HR, RR, and HRV. The ability of the exemplary system to undergo dozens of application cycles was explored to ensure its suitability for month-long monitoring. FIG. 5F shows the mechanical testing used to quantify peel strength. After a month's worth of applications on clean skin, over 50% of adhesive strength remained, as shown in FIG. 5G. Comparing this controlled study to the experimental mean ECG and PPG SNR shown in FIG. 5H, the long-term rise and then fall observed for the ECG SNR may be a combination of initially increasing user proficiency in data collection followed by this adhesive degradation. For PPG, plotted on a narrower vertical scale than ECG, the abrupt shift within the third week of the trial corresponded to roughly +3 dB in mean SNR and may be spurious. Regardless, what signal quality remained after the full duration of the study was sufficient to retain over 95% of the original ECG and PPG signal quality on average.

After the trial, over forty hours of submitted data were processed to assess the exemplary system's ability to detect differences between the high-risk and low-risk populations. Data uploads were uniform throughout the experiment, as shown in FIG. 5I.

FIGS. 5J-5O summarize remote clinical results obtained with wearable devices (i.e., fabricated sensor devices) for differentiating risk levels. Statistical differences were observed between the two groups for HR (as shown in FIG. 5J, p<0.05), in line with known physiological manifestations of cardiovascular stress [19]. Elevated HR (tachycardia) was predictive of cardiovascular for those with preexisting hypertension [20], which was the case for 5 of the 13 high-risk participants and none of the low-risk. The risk posed by tachycardia, as with the danger of postpartum complications, was known to scale with age and can manifest as myocardial infarction and sudden cardiac death from ventricular fibrillation [21]. Though still not entirely understood, the physiological origins of tachycardia and its implications on cardiovascular health were explored in [20a]. Thus, in detecting tachycardia, the exemplary system identified an elevated risk for cardiovascular morbidity in the predicted experimental group.

Separately, HRV, another known predictor of poor cardiovascular outcomes as detailed by Fink in a previous study, was also higher in the high-risk ground (as shown in FIG. 5K, p<0.01). Time domain measures such as the standard deviation of beat-to-beat intervals (shown as HRV) were negatively correlated with higher risk. As reported in [22], reduced HRV may represent impairments in the neural regulation of HR. HRV may also predict the onset of hypertension and myocardial ischemia.

Conversely, however, the high-risk group of this experiment showed elevated HRV. Given the sensitivity of the standard deviation to outliers, the suboptimal fit of the device during the trial, in conjunction with the low sample size for each group of the study, skewed the beat-to-beat timing variances. Skin temperature was also higher in the high-risk group (as shown in FIG. 5L, p<0.001), though a relation between body temperature and cardiovascular risk was not well documented. No significant differences were observed between RR (as shown in FIG. 5M), SpO₂ (as shown in FIG. 5N, p>0.05), and blood pressure (BP) (as shown in FIG. 5O, p>0.05) of high-risk and low-risk groups. Systolic and diastolic pressures between both groups were not different despite the hypertensive diagnoses of nearly half of the high-risk population. First-month postpartum individuals were under physiological and psychological stress as they recuperated from childbirth, which was known to create blood pressure confounders shared by both experimental groups [23].

Calibration-free blood pressure monitoring. Hypertension is a key risk factor for postpartum complications. Standard non-invasive methods in clinical practice for measuring BP rely on cuff-based sphygmomanometers, which disallow continuous measurements and are generally uncomfortable. Conversely, state-of-the-art wearable systems may provide blood pressure through pulse transit time (PTT) regression [24], which relates the propagation speed of the PPG pressure wave to arterial pressure. Table 1 shows various blood pressure prediction models integrated in the state-of-the-art wearable systems.

TABLE 1

| | | | Meeting the AAMI | |
|---|---|---|---|---|
| | | | and FDA Standards | |
| Ref. | Model | Subjects | SBP | DBP |
| This study | Residual CNN | 82 | ✓ | ✓ |
| [24a] | Linear Regression | 21 | — | — |
| [24b] | Linear Regression | 44 | — | ✓ |
| [24c] | Linear Regression | 23 | — | ✓ |
| [24d] | Linear Regression | 3 | ✓ | ✓ |
| [24e] | Linear Regression | 2 | — | — |
| [37] | ANN | 35 | — | — |
| [38] | CNN | 4 | ✓ | ✓ |

PTT methods still required calibration via an invasive arterial line or a time-consuming and less accurate blood pressure cuff. Neither the clinical nor wearable device PTT status quo was ideal for a wearable postpartum monitoring system, which should predict BP continuously and without requiring patients—many of whom already struggled to access clinical resources—to participate in invasive calibration studies. Instead, the study leveraged the public availability of large waveform databases for training a machine learning (ML) model in the exemplary system. Specifically, the study used the Multi-parameter Intelligent Monitoring for Intensive Care (MIMIC) waveform database, which provided synchronized PPG, ECG, BP, and other waveforms from tens of thousands of intensive care unit patients. The study selected 82 subjects with synchronized ABP and BP from the first iteration MIMIC-I subset of the database by prior PPG-ABP machine learning study. This totaled 200 patient days of PPG and BP signals, which were processed with peak detection, bandpass filtering, and segmentation.

To extract as much information as possible from the PPG signal, the study used the continuous wavelet transform (CWT) to generate spectro-temporal data as images of 10-second segments. The chosen machine learning (ML) model, a deep residual convolutional neural network (CNN), reflected this input representation. CNNs were unique for their use of learned convolutional kernels, making them ideal for image processing. The large size of the input images necessitated many convolutional layers with down-sampling to extract all useful information and to reduce the dimensionality of the feature set for the prediction of blood pressure with a final fully connected neural network. However, deeper networks may present differences to learning due to vanishing or exploding gradients, wherein the networks cannot be trained further because the backpropagating signals used to adjust network weights were either very large or almost zero.

Normalization of layer inputs in a process known as batch normalization (i.e., batch norm) may mitigate these issues [25]. However, deep networks leveraging this method may still lose accuracy with increasing depth, a problem addressed by on residual connections. For a given layer, residual or “shortcut” connections may bypass its transformations and add the layer's unchanged input to its transformed output, preserving the features obtained up to that point by, in the extreme case, setting the layer's weights to zero and retaining only the original input. Preserving the identity mapping with each residual connection ensured the network did not both need to retain its learning and advance in the learning task within a single network block. This insight was an important development in the design of deep networks and remained a fixture of the field.

As was standard for deep residual networks, the study developed a repeated block structure in which successive blocks lowered their output resolution but increased the number of output image channels (i.e., feature maps). This implementation detail was critical to the performance of the ML model in the exemplary system, as downsampling increased the receptive field—the region in the input image a filter in a deeper layer of the ML model may learn from—and provided some invariance to transformations of the original image. These outcomes were desirable for real-world scalogram data, where spectral content of interest may shift within a segment or have a meaningful interaction with content elsewhere in that segment.

To achieve this, the study developed a modular network block based on standard residual CNN design to both downsample the incoming feature maps from the previous block and perform its own feature extraction for downstream blocks.

FIGS. 5P-5U show the machine learning model configured for the exemplary system. FIG. 5P shows the residual-convolutional block, developed in the study, configured to double the feature map output while downsampling the input.

FIG. 5Q shows the complete residual-convolutional neural network model configured to predict blood pressure from PPG scalograms. As shown in FIG. 5Q, the network began with an initial large-kernel convolution, followed by three such blocks designed for 64-, 128-, and 256-channel outputs, respectively, with each output therefore downsampled by a factor of 2. Within each block, the study used batch norm and rectified linear unit activations, along with stridden convolutions for downsampling.

Training on the image stack provided from transforming and labeling the MIMIC data subset, the study achieved 4.84±4.16 mmHg systolic (as shown in FIG. 5R) and 2.86±2.97 mmHg diastolic (as shown in FIG. 5S) mean absolute error and standard deviation on the held-out test data, meeting the Association for the Advancement of Medical Instrumentation and U.S. Food and Drug Administration standards for clinic-grade blood pressure prediction (5±8 mmHg). The results shown in FIG. 5T captured this performance, with predicted and true systolic and diastolic pressures closely agreeing for over 40 continuous minutes of test data.

Previous deep learning study achieved lower errors with stateful neural networks [27], but these were performed with smaller population sizes than the instant study's training set of 87 patients. As shown in FIG. 5U, there was an agreement between blood pressure values from the ML model of the exemplary system and the blood pressure values from the blood pressure cuff provided to the patients. The discrepancy between the two types of pressure values shown in FIG. 5U was intra-day differences in timing between the patch and cuff measurements and potentially more fundamental difficulties in predicting blood pressure at the sternum using models trained at the finger. Nonetheless, as summarized in Table 2, the exemplary system and method represented one of the first efforts in leveraging contemporary cloud and machine learning models to serve continuous blood pressure prediction in real-time to at-risk patient groups.

Table 2 shows the blood pressure prediction performance of the exemplary system and the state-of-the-art systems. In Table 2, SBP denotes systolic blood pressure, and DBP denotes diastolic blood pressure.

TABLE 2

| | Clinical Validation | | Telemedicine Technology | | Blood Pressure Prediction | |
|---|---|---|---|---|---|---|
| | Postpartum | Number of | Cloud | Multiplatform | Calibration- | SBP/DBP |
| Ref. | Monitoring | participants | Integration | Mobile App | Free Method | Error |
| This | ✓ | 20 | ✓ | ✓ | ✓ | 4.84 ± 4.16 |
| study | | | | | | 2.86 ± 2.97 |
| [16a] | — | — | ✓ | — | ✓ | Not specified |
| [16b] | — | — | ✓ | — | — | — |
| [30] | — | — | — | — | — | −0.16 ± 2.97 |
| | | | | | | 2.83 ± 1.68 |
| [24d] | — | 576 | — | ✓ | — | 0.6 |
| | | | | | | 0.2 |
| [31] | — | — | ✓ | — | — | — |
| [32] | — | 10 | — | — | — | — |
| [33] | — | — | ✓ | — | — | — |
| [34] | — | — | — | — | — | — |
| [35] | — | — | — | — | — | — |
| [36] | — | — | — | — | — | — |
| [24e] | — | — | ✓ | — | — | — |

### Experiment Protocols in the Study

Before or during the (i) fabrication of the sensor device and (ii) evaluation of the performance of the exemplary system, the study performed a plurality of experiment protocols described below.

Nanomembrane electrodes. Electrode substrate preparation consisting of 15.2 μm polyimide film on polydimethylsiloxane (PDMS)-coated glass base followed the protocol discussed in [13c]. Electron beam deposition was used to evaporate the electrode materials, plating the substrate with high spatial resolution. A 200 nm copper base layer was used to minimize the electrical impedance of the electrodes. A 10 nm chromium adhesion layer and a 100 nm gold layer finalized the deposition process, providing a skin-safe, low-resistance, mechanically robust nanomembrane composite. The electrode pattern was excised from the plated slides using a 1030 nm femtosecond laser, the ultra-short pulse duration of which permitted the creation of a compact electrode geometry by minimizing the imparted thermal loads on the target.

PPG forcing. To optimize the height of the elastomer block, the study inserted a force-sensitive resistor (FSR) (UNEO, GD-03B) between the sensor and chest, and the study measured the force between the PPG sensor and the chest for different elastomer thicknesses. The study measured forcing for blocks with 0-, 2-, 4-, and 6-mm heights. The study performed calibration of the FSR using a Mark-10 ESM303 1.5 kN motorized test stand with a Mark-10 M5-5 25 N force meter and BK891 LCR meter. The calibration procedure involved cycling a sternum analog into the PPG sensor and measuring the relationship between force and resistance. The sternum analog was manufactured by sewing a piece of synthetic skin (SynDaver, Adult Skin 2N) to a rigid plastic backing.

Sensing electronics. The study developed the fPCB for the fabricated sensor device to use an nRF52 microcontroller at a 3.3 V logic level. Interfaced with this via the SPI protocol was a 24-bit ADS1292 analog-to-digital converter (ADC) sampling at 250 Hz for ECG collection. The nanomembrane electrodes interfaced with the fPCB and, thus, the ADC using anisotropic conductive film (ACF). The study collected the PPG via the 18-bit, two-channel MAX30102 sampling red and infrared absorbance at 50 Hz, interfaced with the microcontroller via I2C. Sharing this I2C bus was a TMP117 12-bit temperature sensor sampling at 1 Hz. The fabricated sensor device used a 120 mAh rechargeable battery, which provided nearly 24 hours of continuous monitoring ability at a steady-state current draw of 4 to 5 mA.

Finite element analysis. The study investigated both the electrode pad and interconnect geometries of the fabricated sensor device under load using FEA in Ansys Mechanical (Ansys, Inc.). Given that nearly all the electrode cross sections belonged to the polyimide substrate, the study modeled the electrodes uniformly as 0.01 mm-thick polyimide with isotropic properties, namely elastic modulus E=2.4 GPa, Poisson ratio v=0.4, and density p=1380 kg·m⁻³. The study also investigated the fPCB using the same material properties assuming a uniform thickness of 0.1 mm.

PPG follow-up. The study prepared a postpartum monitor at the sternum and the reference device at the finger for simultaneous measurements to replicate the lag in saturation shown in FIG. 4N. In addition to these, another MAX30102 PPG sensor was placed against a finger on the same hand as the reference device and a toe on the same side of the body as both finger sensors. The study connected all MAX30102 devices simultaneously using timestamp synchronization. The reference device was connected separately to a laptop, and the local system time for the mobile application was calibrated to the National Institute of Standards and Technology servers.

Human subject study. The study recruited twenty pregnant Black women in collaboration with the UIC Nursing School. After delivery, maternal health clinicians classified the participant as high or low-risk based on the individual's medical history. Signed consent forms with written and verbal consent were received by the clinicians before discharge from the hospital in preparation for at-home measurements, which included both the fabricated sensor device described herein and a supplemental blood pressure cuff and finger blood oxygen sensor. Participants received an informational video on expected sensor device usage, which involved streaming data to the cloud for at least 10 minutes each day for their first 28 days after returning home.

Statistical methods. PPG forcing significance proceeded as follows: Levene's test confirmed that the groups exhibited significantly different variances (p<0.01), and so popular analysis of variance (ANOVA) techniques for pairwise significance that assumed homogeneous variance, such as Tukey's range test could not be employed soundly. Games-Howell testing, a nonparametric form of Tukey's range test with no assumptions of normality or variance homogeneity, was used instead under the null hypothesis that all group means were equal. Separately, Welch's t-tests were used to assess differences between the high and low-risk groups in the clinical data of FIG. 5B.

Adhesive strength. The study measured adhesive strength over 28 application cycles using a Mark-10 M5-5 25N force meter. Each cycle consisted of a peel test from cleaned forearm skin at a constant rate of 3 mm·s⁻¹, followed by a measurement session with placement at the sternum for quantifying ECG and PPG SNR. The average force was found for each peel test and normalized by the width of the patch, while SNR was calculated in decibels per Equation 1.

$${SNR}_{db} = 20 {log}_{10} ( \frac{{x}_{signal} ( t )}{{x}_{noise} ( t )} ) \qquad ( Eq . 1 )$$

Multiplatform cloud integration. A mobile application compatible with Android and iOS was developed using the Flutter development framework. Both major mobile operating systems can be targeted with Flutter using a single codebase, reducing development time and project complexity while expanding the scope of the fabricated sensor device to cover nearly all mobile devices currently in consumer use. The study chose the Google Cloud Platform as the cloud provider for its compatibility with the Flutter SDK.

Global time synchronization. Accurate timestamping of received data allowed for direct comparison to data collected by other devices. Still, its implementation was complicated by a lack of obvious ground truth times available to wearable device systems. Mobile device (e.g., tablet and smartphone) system time may be a natural choice, but local time on consumer mobile devices can vary from true time on the order of seconds, an unacceptable error when comparing biosignal waveforms. Though the operating system of these devices periodically synchronizes with true time automatically and may provide the user with the ability to synchronize manually, it may be impossible to derive the synchronization status of a mobile device from its uploaded data alone, making comparisons between data dubious without a deliberate synchronization solution. Thus, while still using the timekeeping convenience provided by system time, the study used the existing internet connection necessary for cloud integration to synchronize the tablet with atomic clocks via the Network Time Protocol (NTP). At application startup and tunable intervals thereafter, the tablet corrected for any local system time deviations from true time by referencing these atomic clock-backed servers. Synchronizing in this way placed the device-timestamped BLE packets on a global timeline shared by any other device with similarly accurate timestamping, such as conventional clinical monitoring equipment or other devices operating within this exemplary system.

Multi-parameter Intelligent Monitoring for Intensive Care (MIMIC) dataset preprocessing. FIG. 5V shows a machine learning preprocessing, training, and evaluation pipeline for the exemplary system, demonstrating interquartile range (IQR) thresholding and peak amplitude average.

The study bandpass filtered PPG from 0.8 to 8 Hz with a fourth-order Butterworth filter. The study then performed peak-finding on the ABP and negative ABP signal for systolic and diastolic peaks, respectively, and the study split both the filtered PPG and its corresponding ABP into 10-second segments without overlap. PPG segments with standard deviations in the interquartile range of each patient's distribution of segment standard deviation were selected; both high-variance and low-variance data were excluded because, as a previous study highlighted [29], real-world biosignals such as these exhibited motion artifacts or periods of signal dropout that manifested as outliers in distributions of variance. Following segmentation and elimination of segments with extreme variance, scalograms were generated via Morlet CWT. In parallel with scalogram generation, peak-finding was performed on the entirety of the patient's ABP waveform and any segments with physiologically unreasonable systolic blood pressure (SBP) and diastolic blood pressure (DBP) such that 80≤SBP≤180 and 50≤DBP≤100 were ignored and the corresponding PPG segments dropped from the dataset. If all ABP peaks were reasonable, their ABP peak amplitudes were averaged to produce a single pair of SBP/DBP labels for that segment. FIG. 5W shows the synchronized waveform processing for machine learning showing ABP (top), ECG (middle), and PPG (bottom). Segments were indicated by vertical bars.

Continuous wavelet transform. The continuous wavelet transformation of a signal x(t) is defined per Equation 2.

$$X ( τ , s ) = {\int }_{- ∞}^{∞} x ( t ) \frac{1}{\sqrt{\vert s \vert}} ψ ( \frac{t - τ}{s} ) dt , ∀ s , τ ∈ ℝ \qquad ( Eq . 2 )$$

In Equation 2, s denotes scales, τ denotes shift, and ψ(t)=exp(jω_{ψ}t)exp(−αt²) for a Morlet Wavelet. The scales chosen for this wavelet, 20 to 140, approximated the passband of 0.8 to 8 Hz, as compared to the previously described MIMIC data bandpass filtering. The generated scalograms were 120×1200 images representing frequency content vertically and time horizontally, which were z-score normalized individually (i.e., normalized with respect to their own mean and variance). FIG. 5X shows the distribution of PPG and ECG scalogram values generated after transforming the input dataset. As shown in FIG. 5X, distributions of each channel's training data are on the right, illustrating the effect of normalization.

Blood pressure machine learning. With the data pipeline and model architecture shown respectively in FIGS. 5P-5V, training proceeded with a learning rate α of 0.001 using the Adam optimizer with momentum set to 0.9 and a batch size of 32. The study chose early stopping at 20 epochs and a weight decay of 0.0005 to mitigate overfitting. Weight decay was a type of regularization in which the L₂ norm of the network's weights was added to the network's function (analogously to ridge regularization in least-squares regression). For a network with weights w and weight decay λ, the loss function [symbol] (x; w) for a batch of size N examples can be defined per Equation 3.

$$ℒ ( x ; w ) = \frac{1}{N} {\sum }_{i = 1}^{N} \frac{1}{2} {( {w}^{T} {x}_{i} + b - {y}_{i} )}^{2} + \frac{λ}{2} {‖ w ‖}_{2}^{2} \qquad ( Eq . 3 )$$

The network complexity itself thus became a point of optimization.

### DISCUSSION

Discussion #1. Noncommunicable diseases (NCDs) have become primary sources of human mortality in the 21st century. The World Health Organization (WHO) estimates that nearly three-quarters of all deaths globally result from NCDs, such as diabetes, pulmonary disorders, cancer, and cardiovascular disease [1], with many proving chronic, debilitating, and ultimately fatal. Lifestyle characteristics play a role in the genesis of these diseases [2], and they have become a challenge to clinical infrastructures focused on acute treatment over prevention and long-term monitoring. Clinical literature has indicated that broadened screening, increased awareness, and improved clinical accessibility are crucial in mitigating these diseases' ongoing and worsening effects [3].

One manifestation of NCDs is postpartum mortality, a complication occurring after childbirth stemming from preexisting cardiovascular diseases (CVDs) [4]. In conjunction with the increasing incidence of CVDs, pregnancy-related mortality rates have increased in the United States for the past three decades, ranking it last among peer nations for maternal outcomes and placing it among the few countries globally in which childbirth-related mortality rates continue to increase in the 21st century [5]. Underlying this tragedy are still higher rates of death among racial and ethnic minority groups, with Black women 2.6 times more likely to die due to pregnancy and giving birth compared to non-Hispanic white women (69.9 deaths per 100,000 births compared to 26.6 deaths per 100,000 as of 2021) [6]. While the causes of this rising maternal mortality and its racial disparities are not understood, the postpartum period—the time beginning after childbirth and lasting for up to 12 months after that—is a window for preventing maternal death; recent public health data show that 40% of pregnancy-related maternal deaths in the United States occur 1 to 42 days postpartum and over 50% of such deaths occur within the first year postpartum [4b], [5]. In line with trends for women, the plurality of these deaths is attributed to CVD, including cardiomyopathy, sudden heart failure, and hypertensive disorders, the latter of which is a common pregnancy-related complication [7]. With the current standard of postpartum care in the United States being a follow-up 4 to 6 weeks after childbirth, many of these lethal postpartum complications occurring in the first month are missed. Adding to this is the ineffectiveness of the follow-up in addressing the needs of minority groups, with recent work finding that African-American and Hispanic women struggle to attend meetings due to deficits in transportation, access, and patient awareness [8]. Thus, the women most vulnerable to NCD complications during the most vulnerable time after childbirth may not receive screening and any necessary interventional care [9].

With recent advances in manufacturing [10], flexible electronics, and telemedicine, soft wearable sensors present compelling opportunities for addressing the challenges posed by NCDs in communities lacking sufficient clinical infrastructure. Wearables, noted for their cost-effectiveness [11], are well-suited to postpartum monitoring given the close coupling of postpartum complications (and CVDs more broadly) with socioeconomic standing [12]. However, soft sensors need not sacrifice measurement fidelity for their accessibility; reducing bulkiness and providing skin conformality compared to conventional rigid alternatives has successfully reduced motion artifacts and improved signal-to-noise ratio for biosignal measurements [13]. While capable of high-quality sensing, the compact and flexible form factor of wearable devices also makes them amenable to long-term, daily monitoring, which is critical given that both comfort and ease of use are deciding factors in the acceptance of wearables by patients across a spectrum of disease groups [14]. Further, the proliferation of wireless protocols such as Bluetooth Low-Energy (BLE), capable of supporting biosignal data throughputs, has made these devices compatible with major categories of consumer devices, including smartphones, tablets, and personal computers, permitting easy integration with technology familiar to patients and used in consumer health monitoring. However, only some reported wearable devices have shown compatibility with long-term, at-home use. Others need a greater breadth of sensors or derived metrics to characterize cardiovascular health, such as blood oxygen saturation or blood pressure. Further, most do not report being compatible with all major consumer mobile devices on the market, limiting their patient applicability. Few have been validated through long-term clinical studies, and none have seen use in postpartum contexts. Mirroring the lack of clinical solutions for increasingly severe postpartum mortality in the United States, there is a marked absence of previous studies suitable for long-term, at-home use in postpartum monitoring.

The instant study introduced a soft, wearable, cloud-interfaced system capable of recording high-quality electrocardiography (ECG), heart rate (HR), heart rate variability (HRV), photoplethysmography (PPG)-based blood oxygen saturation (SpO₂), respiration rate (RR), skin temperature, blood pressure (BP) for postpartum cardiovascular monitoring. The study addressed the limitations in current clinical practice contributing to the severe maternal health crisis in the United States by enabling long-term, remote, continuous, and cost-effective monitoring of key cardiovascular health metrics. Postpartum women, who spend weeks outside the clinic during their most vulnerable time unattended, can continue to recover at home while providing themselves and their clinicians real-time insight into their health, providing a feedback loop for clinical decision-making where none exists. For this improvement over the status quo, the study introduced innovations in the mechanical design of sternum-based wearable sensors, provided real-time delivery of key cardiovascular metrics via a multiplatform mobile application, and integrated machine learning for real-time blood pressure prediction via the cloud. As shown in Table 1, no prior wearable systems validated in a clinical trial have achieved calibration-free blood pressure prediction remotely. In the study, the validation involved high- and low-risk postpartum women in an at-home month-long study, wherein the exemplary system shared daily updates with clinical staff and detected key indicators of elevated postpartum risk, including tachycardia in the designated high-risk group.

Discussion #2. Future studies may develop effective telemedicine for patient groups in low-resource environments. This portable and wearable platform may support additional sensors for detecting glucose, activity, and post-operative signals. Further, cloud integration presents opportunities for algorithmic development in the wearable device space, for example, by expanding on the broad portfolio of modern machine learning models, such as transformers, to include waveform analysis. As “edge” applications (i.e., those with machine learning models hosted on a device) continue to be explored by industry, cloud computation may be replaced in future studies by optimized, fine-tuned local models.

### CONCLUSION

The construction and arrangement of the systems and methods, as shown in the various implementations, are illustrative only. Although only a few implementations have been described in detail in this disclosure, many modifications are possible (e.g., variations in sizes, dimensions, structures, shapes, proportions of the various elements, values of parameters, mounting arrangements, use of materials, colors, orientations, etc.). For example, the position of elements may be reversed or otherwise varied, and the nature or number of discrete elements or positions may be altered or varied. Accordingly, all such modifications are intended to be included within the scope of the present disclosure. The order or sequence of any process or method steps may be varied or re-sequenced according to alternative implementations. Other substitutions, modifications, changes, and omissions may be made in the design, operating conditions, and arrangement of the implementations without departing from the scope of the present disclosure.

The present disclosure contemplates methods, systems, and program products on any machine-readable media for accomplishing various operations. The implementations of the present disclosure may be implemented using existing computer processors, or by a special purpose computer processor for an appropriate system, incorporated for this or another purpose, or by a hardwired system. Implementations within the scope of the present disclosure include program products, including machine-readable media for carrying or having machine-executable instructions or data structures stored thereon. Such machine-readable media can be any available media that can be accessed by a general-purpose or special-purpose computer or other machine with a processor. By way of example, such machine-readable media can comprise RAM, ROM, EPROM, EEPROM, CD-ROM or other optical disk storage, magnetic disk storage or other magnetic storage devices, or any other medium which can be used to carry or store desired program code in the form of machine-executable instructions or data structures, and which can be accessed by a general purpose or special purpose computer or other machine with a processor.

When information is transferred or provided over a network or another communications connection (either hardwired, wireless, or a combination of hardwired or wireless) to a machine, the machine properly views the connection as a machine-readable medium; thus, any such connection is properly termed a machine-readable medium. Combinations of the above are also included within the scope of machine-readable media. Machine-executable instructions include, for example, instructions and data that cause a general-purpose computer, special-purpose computer, or special-purpose processing machine to perform a certain function or group of functions.

Although the figures show a specific order of method steps, the order of the steps may differ from what is depicted. Also, two or more steps may be performed concurrently or with partial concurrence. Such variation will depend on the software and hardware systems chosen and on the designer's choice. All such variations are within the scope of the disclosure. Likewise, software implementations could be accomplished with programming techniques with rule-based logic and other logic to accomplish the various connection steps, processing steps, comparison steps, and decision steps.

Machine Learning. In addition to the machine learning features described above, the analysis system can be implemented using one or more artificial intelligence and machine learning operations. The term “artificial intelligence” can include any technique that enables one or more computing devices or computing systems (i.e., a machine) to mimic human intelligence. Artificial intelligence (AI) includes but is not limited to knowledge bases, machine learning, representation learning, and deep learning. The term “machine learning” is defined herein to be a subset of AI that enables a machine to acquire knowledge by extracting patterns from raw data. Machine learning techniques include, but are not limited to, logistic regression, support vector machines (SVMs), decision trees, Naïve Bayes classifiers, and artificial neural networks. The term “representation learning” is defined herein to be a subset of machine learning that enables a machine to automatically discover representations needed for feature detection, prediction, or classification from raw data. Representation learning techniques include, but are not limited to, autoencoders and embeddings. The term “deep learning” is defined herein to be a subset of machine learning that enables a machine to automatically discover representations needed for feature detection, prediction, classification, etc., using layers of processing. Deep learning techniques include but are not limited to artificial neural networks or multilayer perceptron (MLP).

An artificial neural network (ANN) is a computing system including a plurality of interconnected neurons (e.g., also referred to as “nodes”). This disclosure contemplates that the nodes can be implemented using a computing device (e.g., a processing unit and memory as described herein). The nodes can be arranged in a plurality of layers, such as an input layer, an output layer, and optionally one or more hidden layers with different activation functions. An ANN having hidden layers can be referred to as a deep neural network or multilayer perceptron (MLP). Each node is connected to one or more other nodes in the ANN. For example, each layer is made of a plurality of nodes, where each node is connected to all nodes in the previous layer. The nodes in a given layer are not interconnected with one another, i.e., the nodes in a given layer function independently of one another. As used herein, nodes in the input layer receive data from outside of the ANN, nodes in the hidden layer(s) modify the data between the input and output layers, and nodes in the output layer provide the results. Each node is configured to receive an input, implement an activation function (e.g., binary step, linear, sigmoid, tanh, or rectified linear unit (ReLU) function), and provide an output in accordance with the activation function. Additionally, each node is associated with a respective weight. ANNs are trained with a dataset to maximize or minimize an objective function. In some implementations, the objective function is a cost function, which is a measure of the ANN's performance (e.g., error such as L1 or L2 loss) during training, and the training algorithm tunes the node weights and/or bias to minimize the cost function. This disclosure contemplates that any algorithm that finds the maximum or minimum of the objective function can be used for training the ANN. Training algorithms for ANNs include but are not limited to backpropagation. It should be understood that an artificial neural network is provided only as an example machine learning model. This disclosure contemplates that the machine learning model can be any supervised learning model, semi-supervised learning model, or unsupervised learning model. Optionally, the machine learning model is a deep learning model. Machine learning models are known in the art and are therefore not described in further detail herein.

A convolutional neural network (CNN) is a type of deep neural network that has been applied, for example, to image analysis applications. Unlike traditional neural networks, each layer in a CNN has a plurality of nodes arranged in three dimensions (width, height, depth). CNNs can include different types of layers, e.g., convolutional, pooling, and fully-connected (also referred to herein as “dense”) layers. A convolutional layer includes a set of filters and performs the bulk of the computations. A pooling layer is optionally inserted between convolutional layers to reduce the computational power and/or control overfitting (e.g., by downsampling). A fully-connected layer includes neurons, where each neuron is connected to all of the neurons in the previous layer. The layers are stacked similarly to traditional neural networks. GCNNs are CNNs that have been adapted to work on structured datasets such as graphs.

Other Supervised Learning Models. A logistic regression (LR) classifier is a supervised classification model that uses the logistic function to predict the probability of a target, which can be used for classification. LR classifiers are trained with a data set (also referred to herein as a “dataset”) to maximize or minimize an objective function, for example, a measure of the LR classifier's performance (e.g., an error such as L1 or L2 loss), during training. This disclosure contemplates that any algorithm that finds the minimum of the cost function can be used. LR classifiers are known in the art and are therefore not described in further detail herein.

A Naïve Bayes' (NB) classifier is a supervised classification model that is based on Bayes' Theorem, which assumes independence among features (i.e., the presence of one feature in a class is unrelated to the presence of any other features). NB classifiers are trained with a data set by computing the conditional probability distribution of each feature given a label and applying Bayes' Theorem to compute the conditional probability distribution of a label given an observation. NB classifiers are known in the art and are therefore not described in further detail herein.

A k-NN classifier is an unsupervised classification model that classifies new data points based on similarity measures (e.g., distance functions). The k-NN classifiers are trained with a data set (also referred to herein as a “dataset”) to maximize or minimize a measure of the k-NN classifier's performance during training. This disclosure contemplates any algorithm that finds the maximum or minimum. The k-NN classifiers are known in the art and are therefore not described in further detail herein.

A majority voting ensemble is a meta-classifier that combines a plurality of machine learning classifiers for classification via majority voting. In other words, the majority voting ensemble's final prediction (e.g., class label) is the one predicted most frequently by the member classification models. The majority voting ensembles are known in the art and are therefore not described in further detail herein.

It is to be understood that the methods and systems are not limited to specific synthetic methods, specific components, or to particular compositions. It is also to be understood that the terminology used herein is for the purpose of describing particular implementations only and is not intended to be limiting.

As used in the specification and the appended claims, the singular forms “a,” “an” and “the” include plural referents unless the context clearly dictates otherwise. Ranges may be expressed herein as from “about” one particular value, and/or to “about” another particular value. When such a range is expressed, another implementation includes from the one particular value and/or to the other particular value. Similarly, when values are expressed as approximations, by use of the antecedent “about,” it will be understood that the particular value forms another implementation. It will be further understood that the endpoints of each of the ranges are significant both in relation to the other endpoint, and independently of the other endpoint.

“Optional” or “optionally” means that the subsequently described event or circumstance may or may not occur and that the description includes instances where said event or circumstance occurs and instances where it does not.

Throughout the description and claims of this specification, the word “comprise” and variations of the word, such as “comprising” and “comprises,” means “including but not limited to,” and is not intended to exclude, for example, other additives, components, integers or steps. “Exemplary” means “an example of” and is not intended to convey an indication of a preferred or ideal implementation. “Such as” is not used in a restrictive sense but for explanatory purposes.

Disclosed are components that can be used to perform the disclosed methods and systems. These and other components are disclosed herein, and it is understood that when combinations, subsets, interactions, groups, etc. of these components are disclosed while specific reference of each various individual and collective combinations and permutation of these may not be explicitly disclosed, each is specifically contemplated and described herein, for all methods and systems. This applies to all aspects of this application, including, but not limited to, steps in disclosed methods. Thus, if there are a variety of additional steps that can be performed it is understood that each of these additional steps can be performed with any specific implementation or combination of implementations of the disclosed methods.

The following patents, applications, and publications, as listed below and throughout this document, are hereby incorporated by reference in their entirety herein.
[1] World Health Organization-Newsroom Report-fact sheets, 2023.
[2] R. Arena, C. J. Lavie, M,-F. Hivert, M. A. Williams, P. D. Briggs, M. Guazzi, Expert Rev. Cardiovasc. Ther. 2016, 14, 15.
[3] a) K. Strong, N. Wald, A. Miller, A. Alwan, W. C. Group, J. Med. Screen. 2005, 12, 12; b) S. H. Habib, S. Saha, Clin. Res. Rev. 2010, 4, 41.
[4] a) M. F. Mogos, J. L. Salemi, K. K. Spooner, B. L. McFarlin, H. H. Salihu, J. Hypertens. 2018, 36, 608; b) E. E. Petersen, N. L. Davis, D. Goodman, S. Cox, N. Mayes, E. Johnston, C. Syverson, K. Seed, C. K. Shapiro-Mendoza, W. M. Callaghan, Morb. Mortal. Weekly Rep. 2019, 68, 423.
[5] a) N. Hamilton, N. Stevens, T. Lillis, N. Adams, The Fourth Trimester: Toward Improved Postpartum Health and Healthcare of Mothers and Their Families in the United States, Springer, Berlin, Heidelberg 2018, Vol. 41, pp. 571-576; b) D. Vilda, M. Wallace, L. Dyer, E. Harville, K. Theall, SSM-Populat. Health 2019, 9, 100477.
[6] D. L. Hoyert, Maternal mortality rates in the United States, 2022.
[7] M. C. Gongora, N. K. Wenger, Int. J. Mol. Sci. 2015, 16, 23905.
[8] E. A. Howell, Clin. Obstet. Gynecol. 2018, 61, 387.
[9] M. F. Mogos, K. L. Liese, P. D. Thornton, T. A. Manuck, W. D. O'Brien Jr, B. L. McFarlin, Nurs. Res. 2020, 69, 42.
[10] a) J. Kim, A. Bayro, J. Lee, I. Soltis, M. Kim, H. Jeong, W,-H. Yeo, Biosens. Bioelectron.: X 2023, 14, 100343; b) S. Kwon, H. S. Kim, K. Kwon, H. Kim, Y. S. Kim, S. H. Lee, Y,-T. Kwon, J,-W. Jeong, L. M. Trotti, A. Duarte, Sci. Adv. 2023, 9, eadg9671; c) H. Kim, Y. J. Lee, G. Byun, C. Choi, W. H. Yeo, Adv. Electron. Mater. 2023, 9, 2201294; d) J. Matthews, J. Kim, W. H. Yeo, Anal. Sens. 2023, 3, e202200062.
[11] C. C. Nachiar, N. Ambika, R. Moulika, R. Poovendran, in 2020 Fourth Int. Conf. onI-SMAC (IoT in Social, Mobile, Analytics, and Cloud) (I-SMAC) IEEE, 2020, 1289-1292.
[12] a) A. M. Clark, M. DesMeules, W. Luo, A. S. Duncan, A. Wielgosz, Nat. Rev. Cardiol. 2009, 6, 712; b) E. P. Havranek, M. S. Mujahid, D. A. Barr, I. V. Blair, M. S. Cohen, S. Cruz-Flores, G. Davey-Smith, C. R. Dennison-Himmelfarb, M. S. Lauer, D. W. Lockwood, Circulation 2015, 132, 873.
[13] a) H. Kim, E. Kim, C. Choi, W,-H. Yeo, Micromachines 2022, 13, 629; b) J. Kim, P. Kantharaju, H. Yi, M. Jacobson, H. Jeong, H. Kim, J. Lee, J. Matthews, N. Zavanelli, H. Kim, npj Flexible Electron. 2023, 7, 3; c) Y. S. Kim, J. Kim, R. Chicas, N. Xiuhtecutli, J. Matthews, N. Zavanelli, S. Kwon, S. H. Lee, V. S. Hertzberg, W. H. Yeo, Adv. Healthcare Mater. 2022, 11, 2200170; d) N. Rodeheaver, R. Herbert, Y. S. Kim, M. Mahmood, H. Kim, J. W. Jeong, W. H. Yeo, Adv. Funct. Mater. 2021, 31, 2104070; e) N. Zavanelli, H. Kim, J. Kim, R. Herbert, M. Mahmood, Y,-S. Kim, S. Kwon, N. B. Bolus, F. B. Torstrick, C. S. Lee, Sci. Adv. 2021, 7, eabl4146.
[14] Y. Gao, H. Li, Y. Luo, Ind. Manag. Data Syst. 2015, 115, 1704.
[15] Bureau of the Census, American Community Survey. U. S. Department of Commerce, Ed.: 2021.
[16] a) D. T. Phan, C. H. Nguyen, T. D. P. Nguyen, L. H. Tran, S. Park, J. Choi, B.-I. Lee, J. Oh, Biosensors 2022, 12, 139; b) M. Shao, Z. Zhou, G. Bin, Y. Bai, S. Wu, Sensors 2020, 20, 606.
[17] a) M. Elgendi, Bioengineering 2016, 3, 21; b) S. Rahman, C. Karmakar, I. Natgunanathan, J. Yearwood, M. Palaniswami, J. R. Soc., Interface 2022, 19, 20220012.
[18] J. A. Fan, W,-H. Yeo, Y. Su, Y. Hattori, W. Lee, S,-Y. Jung, Y. Zhang, Z. Liu, H. Cheng, L. Falgout, Nat. Commun. 2014, 5, 3266.
[19] F. Al-Rashed, S. Sindhu, A. Al Madhoun, Z. Ahmad, D. AlMekhled, R. Azim, S. Al-Kandari, M. A.-A. Wahid, F. Al-Mulla, R. Ahmad, Sci. Rep. 2021, 11, 13883.
[20] a) P. Palatini, S. Julius, Clin. Exp. Hypertens. 2004, 26, 637; b) C. Perret-Guillaume, L. Joly, A. Benetos, Prog. Cardiovasc. Dis. 2009, 52, 6.
[21] W. S. Aronow, C. Ahn, A. D. Mercando, S. Epstein, Am. J. Cardiol. 1996, 78, 1175.
[22] A. M. Fink, Int. J. Occup. Med. Environ. Health 2020, 33, 409.
[23] M. Sanghavi, J. D. Rutherford, Circulation 2014, 130, 1003.
[24] a) V. G. Ganti, A. M. Carek, B. N. Nevius, J. A. Heller, M. Etemadi, O. T. Inan, IEEE J. Biomed. Health Inform. 2020, 25, 1926; b) R. Lazazzera, Y. Belhaj, G. Carrault, Sensors 2019, 19, 2557; c) C. Liu, J. T. Kim, S. S. Kwak, A. Hourlier-Fargette, R. Avila, J. Vogl, A. Tzavelis, H. U. Chung, J. Y. Lee, D. H. Kim, Adv. Healthcare Mater. 2021, 10, 2100383; d) D. Ryu, D. H. Kim, J. T. Price, J. Y. Lee, H. U. Chung, E. Allen, J. R. Walter, H. Jeong, J. Cao, E. Kulikova, Proc. Natl. Acad. Sci. USA 2021, 118, e2100466118; e) T. Wu, F. Wu, C. Qiu, J,-M. Redoute, M. R. Yuce, IEEE Int. Things J. 2020, 7, 6932.
[25] A. Veit, M. J. Wilber, S. Belongie, Adv. Neur. Inform. Proc. Syst. 2016, 29.
[26] K. He, X. Zhang, S. Ren, J. Sun, in Proceedings of the IEEE Conf. on Computer Vision and Pattern Recognition, IEEE, New York 2016, 770.
[27] M. S. Tanveer, M. K. Hasan, Biomed. Signal Process. Control 2019, 51, 382.
[28] S. Sundrani, J. Chen, B. T. Jin, Z. S. H. Abad, P. Rajpurkar, D. Kim, NPJ Digit Med. 2023, 6, 60.
[29] C. El-Hajj, P. A. Kyriacou, Biomed. Signal Process. Control 2020, 58, 101870.
[30] T.-W. Wang, H.-W. Chu, W.-X. Chen, Y.-T. Shih, P.-C. Hsu, H.-M. Cheng, S.-F. Lin, IEEE Access 2020, 8, 184909.
[31] N. Glazkova, T. Podladchikova, R. Gerzer, D. Stepanova, Acta Astro-naut. 2020, 166, 613.
[32] M. Klum, M. Urban, T. Tigges, A,-G. Pielmus, A. Feldheiser, T. Schmitt, R. Orglmeister, Sensors 2020, 20, 2033.
[33] S. Murali, F. Rincon, T. Cassina, S. Cook, J.-J. Goy, JMIR Biomed. En-gineer. 2020, 5, e18158.
[34] M. Chan, V. G. Ganti, J. A. Heller, C. A. Abdallah, M. Etemadi, O. T. Inan, Biosensors 2021, 11, 521.
[35] W. Choi, S.-H. Kim, W. Lee, S.-H. Kang, C.-H. Yoon, T.-J. Youn, I.-H. Chae, J Korean Med. Sci. 2020, 35, 1146204.
[36] N. Kant, G. M. Peters, B. J. Voorthuis, C. G. Groothuis-Oudshoorn, M. V. Koning, B. P. Witteman, M. Rinia-Feenstra, C. J. Doggen, J Clin. Monitor. Comput. 2022, 36, 1449.
[37] J. H. Moon, M.-K. Kang, C.-E. Choi, J. Min, H.-Y. Lee, S. Lim, Sci. Rep. 2020, 10, 19015.
[38] B. Ibrahim, R. Jafari, Sci. Rep. 2022, 12, 319.

What is claimed:

1. A system (e.g., without arterial line and pressure cuff) comprising:
an elastomeric substrate having a first side configured to be placed in contact with a skin region of a user (e.g., having a size to place to a substantial portion of a sternum) for a period of time of at least one day, the elastomeric substrate formed of one or more layers and having defined a sensor port at a first sensor region;
a flexible photoplethysmographic circuit assembly configured with photodiodes, a photoplethysmographic circuit, and a forcing membrane, the flexible photoplethysmographic circuit assembly being configured to measure photoplethysmographic (PPG), wherein the flexible circuit assembly is positioned on the elastomeric substrate such that the photodiodes are contact-able to the skin region at the sensor port for the sensor region, and wherein the forcing membrane is in mechanical contact (e.g., directly or indirectly over a tuning spring) and positioned over the photodiodes to urge the photodiodes toward the skin region;
a stretchable electrode array assembly formed in the elastomeric substrate comprising a stretchable electrode array, configured to contact the skin region of the user at a second sensor region, the stretchable electrode array assembly being configured to measure electrocardiographic (ECG), wherein the electrode array is formed by one or more conformable electrodes having a meandering pattern configured to be in a first meandering configuration when placed on the skin and a second meandering configuration when stretched from the first meandering configuration; and
a controller operatively coupled to the flexible photoplethysmographic circuit assembly and the stretchable electrode array assembly (e.g., directly or through a network), the controller having:
a processor; and
a memory having instructions stored thereon, wherein execution of the instructions causes the processor to:
receive, by the processor, measured ECG and PPG signals;
determine, via a trained AI model, estimated blood pressure for the period of time of at least one day using the measured ECG and PPG signals as input to the trained AI model; and
determine heart rate parameter, respiration rate parameter, heart rate variability parameter, and blood oxygen saturation parameter, from the measured ECG and/or PPG signals for the period of time of at least one day,
wherein the determined heart rate parameter, respiration rate parameter, heart rate variability parameter, blood oxygen saturation parameter, and estimated blood pressure are outputted to provide a prolonged vital sign monitor for the user for the period of time of at least one day.

2. The system of claim 1, wherein the controller is physically coupled to the electrode array assembly and the flexible photoplethysmographic circuit in a single integrated sensor-controller device.

3. The system of claim 1, wherein the controller is implemented in a mobile device (e.g., tablet, smartphone) comprising a network interface configured to communicatively operate with the electrode array assembly and the flexible photoplethysmographic circuit through a network.

4. The system of claim 1, wherein the controller is a remote computing device located in a cloud infrastructure comprising a network interface configured to communicatively operate with the electrode array assembly and the flexible photoplethysmographic circuit through a network.

5. The system of claim 1, wherein the first side of the elastomeric substrate has an adhesive.

6. The system of claim 1, wherein the one or more conformable electrodes are formed of (i) a serpentine-patterned structure at a first end and (ii) a terminal at a second end.

7. The system of claim 6, wherein each serpentine-patterned structure of the one or more conformable electrodes is formed of a first layer comprising a metal and a second layer comprising a polyimide.

8. The system of claim 1, wherein the determined heart rate parameter, respiration rate parameter, heart rate variability parameter, blood oxygen saturation parameter, and estimated blood pressure are employed for ambulatory care monitoring.

9. The system of claim 1, wherein the determined heart rate parameter, respiration rate parameter, heart rate variability parameter, blood oxygen saturation parameter, and estimated blood pressure are employed for health monitoring (e.g., postpartum monitoring).

10. The system of claim 1, wherein the trained AI model comprises a neural network.

11. The system of claim 1, wherein the respiration rate parameter is determined from an amplitude modulation operation of R-peaks in the measured ECG signal.

12. A method comprising:
receiving, by a processor, measured ECG and PPG signals from a stretchable electrode array assembly and a flexible photoplethysmographic circuit assembly, respectively;
determining, via a trained AI model, estimated blood pressure for a period of time of at least one day using the measured ECG and PPG signals as input to the trained AI model; and
determining heart rate parameter, respiration rate parameter, heart rate variability parameter, and blood oxygen saturation parameter, from the measured ECG and/or PPG signals for the period of time of at least one day,
wherein the determined heart rate parameter, respiration rate parameter, heart rate variability parameter, blood oxygen saturation parameter, and estimated blood pressure are outputted to provide a prolonged vital sign monitor for a user for the period of time of at least one day.

13. The method of claim 12, wherein one or more conformable electrodes of the stretchable electrode array assembly are formed of (i) a serpentine-patterned structure at a first end and (ii) a terminal at a second end.

14. The method of claim 13, wherein each serpentine-patterned structure of the one or more conformable electrodes is formed of a first layer comprising a metal and a second layer comprising a polyimide.

15. The method of claim 12, wherein the determined heart rate parameter, respiration rate parameter, heart rate variability parameter, blood oxygen saturation parameter, and estimated blood pressure are employed for ambulatory care monitoring.

16. The method of claim 12, wherein the determined heart rate parameter, respiration rate parameter, heart rate variability parameter, blood oxygen saturation parameter, and estimated blood pressure are employed for health monitoring (e.g., postpartum monitoring).

17. The method of claim 12, wherein the trained AI model comprises a neural network.

18. The method of claim 12, wherein the respiration rate parameter is determined from an amplitude modulation operation of R-peaks in the measured ECG signal.

19. A non-transitory computer-readable medium having instructions stored thereon, wherein execution of the instructions by a processor causes the processor to:
receive, by a processor, measured ECG and PPG signals, measured ECG and PPG signals from a stretchable electrode array assembly and a flexible photoplethysmographic circuit assembly, respectively;
determine, via a trained AI model, estimated blood pressure for a period of time of at least one day using the measured ECG and PPG signals as input to the trained AI model; and
determine heart rate parameter, respiration rate parameter, heart rate variability parameter, and blood oxygen saturation parameter, from the measured ECG and/or PPG signals for the period of time of at least one day,
wherein the determined heart rate parameter, respiration rate parameter, heart rate variability parameter, blood oxygen saturation parameter, and estimated blood pressure are outputted to provide a prolonged vital sign monitor for a user for the period of time of at least one day.

20. The non-transitory computer-readable medium of claim 19, wherein one or more conformable electrodes of the stretchable electrode array assembly are formed of (i) a serpentine-patterned structure at a first end and (ii) a terminal at a second end.
